SEQUENCE LISTING (32 sequences)

SEQ ID NO: 1 - protein, 272 aa, Vaccinia virus
MRYIIILAVLFINSIHAKITSYKFESVNFDSKIEWTGDGLYNISLKNYGIKTWQTMYTNVPEGTYDISAFPKNDFVSFWVKFEQGDYKVEEYCTGLCVEVKIGPPTVTLTEYDDHINLYIEHPYATRGSKKIPIYKRGDMCDIYLLYTANFTFGDSEEPVTYDIDDYDCTSTGCSIDFATTEKVCVTAQGATEGFLEKITPWSSEVCLTPKKNVYTCAIRSKEDVPNFKDKMARVIKRKFNKQSQSYLTKFLGSTSNDVTTFLSMLNLTKYS

SEQ ID NO: 2 - DNA, 819 bp, Vaccinia virus
  source 1..819: mol_type = other DNA
atgagatatattataattctcgcagttttgttcattaatagtatacacgctaaaataactagttataagtttgaatccgtcaattttgattccaaaattgaatggactggggatggtctatacaatatatcccttaaaaattatggcatcaagacgtggcaaacaatgtatacaaatgtaccagaaggaacatacgacatatccgcatttccaaagaatgatttcgtatctttctgggttaaatttgaacaaggcgattataaagtggaagagtattgtacgggactatgcgtcgaagtaaaaattggaccaccgactgtaacattgactgaatacgacgaccatatcaatttgtacatcgagcatccgtatgctactagaggtagcaaaaagattcctatttacaaacgcggtgacatgtgtgatatctacttgttgtatacggctaacttcacattcggagattctgaagaaccagtaacatatgatatcgatgactacgattgcacgtctacaggttgcagcatagactttgccacaacagaaaaagtgtgcgtgacagcacagggagccacagaagggtttctcgaaaaaattactccatggagttcggaagtatgtctgacacctaaaaagaatgtatatacatgtgcaattagatccaaagaagatgttcccaatttcaaggacaaaatggccagagttatcaagagaaaatttaataaacagtctcaatcttatttaactaaatttctcggtagcacatcaaatgatgttaccacttttcttagcatgcttaacttgactaaatattcataa

SEQ ID NO: 3 - protein, 353 aa, Vaccinia virus
MTMKMMVHIYFVSLSLLLLLFHSYAIDIENEITEFFNKMRDTLPAKDSKWLNPACMFGGTMNDMATLGEPFSAKCPPIEDSLLSHRYKDYVVKWERLEKNRRRQVSNKRVKHGDLWIANYTSKFSNRRYLCTVTTKNGDCVQGIVRSHIKKPPSCIPKTYELGTHDKYGIDLYCGILYAKHYNNITWYKDNKEINIDDIKYSQTGKELIIHNPELEDSGRYDCYVHYDDVRIKNDIVVSRCKILTVIPSQDHRFKLILDPKINVTIGEPANITCTAVSTSLLIDDVLIEWENPSGWLIGFDFDVYSVLTSRGGITEATLYFENVTEEYIGNTYKCRGHNYYFEKTLTTTVVLE

SEQ ID NO: 4 - DNA, 1062 bp, Vaccinia virus
  source 1..1062: mol_type = unassigned DNA
atgacgatgaaaatgatggtacatatatatttcgtatcattatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatggccactctaggagagccattcagtgcaaagtgtcctcctattgaagacagtcttttatcgcacagatataaagactatgtggttaaatgggagaggctagaaaagaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattaaaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccgaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtatcaccgaggcgaccttgtactttgaaaatgttactgaagaatatataggtaatacatataaatgtcgtggacacaactattattttgaaaaaacccttacaactacagtagtattggagtaa

SEQ ID NO: 5 - protein, 224 aa, Vaccinia virus
MVKNNKISNSCRMIMSTNPNNILMRHLKNLTDDEFKCIIHRSSDFLYLSDSDYTSITKETLVSEIVEEYPDDCNKILAIIFLVLDKDIDVDIETKLKPKPAVRFAILDKMTEDIKLTDLVRHYFRYIEQDIPLGPLFKKIDSYRTRAINKYSKELGLATEYFNKYGHLMFYTLPIPYNRFFCRNSIGFLAVLSPTIGHVKAFYKFIEYVSIDDRRKFKKELMSK

SEQ ID NO: 6 - DNA, 675 bp, Vaccinia virus
  source 1..675: mol_type = unassigned DNA
atggtgaaaaataataaaataagtaatagctgccgaatgataatgagtactaaccctaataatattctaatgagacatctcaaaaatcttacagatgatgaatttaaatgtattattcatcgatcatctgattttctttatttgtccgatagtgactatactagtataaccaaagaaacattagttagtgagatcgtagaagaatatccggatgattgtaataaaatattagctattatatttttggtgttagataaagacatagatgtagatatagaaactaaactaaagcctaaacccgcagttagatttgccattctagacaagatgactgaggatattaaactaacggatctagtcagacattattttagatacatagaacaagatataccactaggtccgttgttcaaaaaaatagattcgtacagaacaagagccattaataagtattcgaaagagttaggattggctactgaatactttaataagtacggacatttaatgttttatactctccctataccatataacagattcttttgtagaaattcgataggctttttagcggttctatcgcctacgataggacacgtaaaagcattttataaattcatagaatatgtttctatagatgatagacgcaaatttaaaaaggaattaatgtcgaaatga

SEQ ID NO: 7 - protein, 512 aa, Vaccinia virus
MESVIFSINGEIIQVNKEIITASPYNFFKRIQDHHLKDEAIILNGINYHAFESLLDYMRWKKINITINNVEMILVAAVIIDVPPVVDLCVKTMIHNINSTNCIRMFNFSKRYGIKKLYNASMSEIINNITAVTSDPEFGKLSKDELTTILSHEDVNVNHEDVTAMILLKWIHKNPNDVDIINILHPKFMTNTMRNAISLLGLTISKSTKPVTRNGIKHNIVVIKNSDYISTITHYSPRTEYWTIVGNTDRQFYNANVLHNCLYIIGGMINNRHVYSVSRVDLETKKWKTVTNMSSLKSEVSTCVNDGKLYVIGGLEFSISTGVAEYLKHGTSKWIRLPNLITPRYSGASVFVNDDIYVMGGVYTTYEKYVVLNDVECFTKNRWIKKSPMPRHHSIVYAVEYDGDIYVITGITHETRNYLYKYIVKEDKWIELYMYFNHVGKMFVCSCGDYILIIADAKYEYYPKSNTWNLFDMSTRNIEYYDMFTKDETPKCNVTHKSLPSFLSNCEKQFLQ

SEQ ID NO: 8 - DNA, 1539 bp, Vaccinia virus
  source 1..1539: mol_type = unassigned DNA
atggaaagcgtgatattttctatcaatggggaaattatacaagtgaataaggaaattattacggcgtctccgtataatttttttaaacgcattcaggatcaccatctaaaggatgaagcgattatattgaatggtataaactatcacgcgtttgaatcgctattagactatatgcgctggaagaagataaacatcaccataaacaatgtagaaatgatactagttgctgccgtaataattgatgttccgcctgtagtagatctatgtgtaaaaactatgattcataatattaattccacaaattgtataaggatgtttaacttttctaaaagatatggaattaaaaaactatataatgcgtcgatgtcagaaataatcaacaatattactgcggtgacatccgatccagaatttggaaaattatcaaaggatgaactgacaactatcttatcccacgaagacgttaacgtaaatcatgaggatgttacagctatgatattattaaagtggatacataaaaatccaaacgatgtagatatcatcaacattttacatcccaagtttatgactaatactatgcgcaatgctatatcattgttgggattaactatatccaaatctacaaagccagtgacacgaaatggtataaaacataatatagtagtcattaaaaactctgattatatatccacaataacccattactctcctaggacagaatattggacgatagtcggtaatacagatagacaattctataatgcaaatgttttacataattgtctatacattattggcggcatgattaacaatagacatgtttattccgtatcgcgggtagatcttgaaacgaaaaaatggaaaacggttactaatatgtcgtcgttaaaatcagaagttagtacttgtgttaacgatggaaagttatatgtaataggaggattagaattttctatttcaacgggtgtggcagaatatttgaaacacggcacttcgaaatggataagacttccaaacttaattactcctagatattcaggcgcgtcggtattcgtaaacgatgatatatatgtaatgggtggagtttataccacgtatgaaaaatatgtagtattaaacgatgtggaatgtttcactaaaaatcgttggataaaaaagtctcccatgcctagacatcatagtatagtttatgctgtagagtacgacggcgacatctatgtaattactggaattactcacgagactcgtaattatctatacaaatatatagttaaggaagacaaatggatagaattgtacatgtactttaaccatgtaggaaagatgttcgtgtgttcttgcggtgattatatcttaattatagcagatgcaaaatacgaatattatccaaaatcaaatacttggaatttgttcgatatgtcaactcgtaatatcgaatattatgatatgtttactaaagatgagactccaaagtgtaacgtaactcataagtcactgccatcgtttttgagcaactgtgaaaaacaatttctacaatag

SEQ ID NO: 9 - protein, 263 aa, Vaccinia virus
MKVESVTFLTLLGIGCVLSCCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 10 - DNA, 792 bp, Vaccinia virus
  source 1..792: mol_type = unassigned DNA
atgaaggtggagagcgtgacgttcctgacattgttgggaataggatgcgttctatcatgctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttttataccgatggaagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgctaa

SEQ ID NO: 11 - protein, 316 aa, Vaccinia virus
MDTIKIFNHGEFDTIRNELVNLLKVVKWNTINSNVTVSSTDTIDISDCIREILYKQFKNVRNIEVSSDISFIKYNRFNDTTLTDDNVGYYLVIYLNRTKSVKTLIYPTPETVITSSEDIMFSKSLNFRFENVKRDYKLVMCSISLTYKPSICRIQYDNNKYLDISDSQECNNICYCVITMDPHHLIDLETICVLVDKSGKCLLVNEFYIRFRKNHIYNSFADLCMDHIFELPNTKELFTLRNDDGRNIAWDNDKLESGNNTWIPKTDDEYKFLSKLMNIAKFNNTKFDYYVLVGDTDPCTVFTFKVTKYYINLNYE

SEQ ID NO: 12 - DNA, 951 bp, Vaccinia virus
  source 1..951: mol_type = unassigned DNA
atggatactattaaaatatttaatcatggagagtttgatactattagaaatgaattggtcaacttgttaaaagttgtaaaatggaacaccattaattcaaatgtaacggtatcgtctaccgacacaatagatatatctgattgtatcagagagatattatacaaacagtttaaaaatgtacgaaatatagaggtgagcagtgatatatctttcataaaatataatagatttaatgatactactctaacagatgataatgtgggatattatttagtgatttatttgaatcggacgaaatctgtaaagactttaatatatcctactccagaaacagtgataacatcatccgaggatataatgttttctaaatctcttaactttagattcgagaatgtaaagcgcgactataaattggttatgtgttctatatctttgacatacaagccatctatatgcagaatccaatacgataacaataaatatttagatattagcgacagtcaagaatgtaataatatatgttactgcgtcataactatggatccccaccacttgatagatttagaaaccatatgtgtcttggtcgataaatcaggaaagtgtttgctagtaaacgagttttatattagatttaggaaaaatcacatctacaatagctttgccgatctatgcatggatcatatatttgaactaccgaatacaaaagaattattcactctgcgcaacgatgatggacgaaacatagcctgggataatgataagttggaaagtggtaataatacatggattcctaaaacagatgatgagtataagtttctatctaaattgatgaatattgcgaagtttaacaacaccaaatttgattactacgtgcttgttggagatacggatccgtgtactgtctttacgttcaaagtaacaaagtattacatcaatctcaattatgaatag

SEQ ID NO: 13 - protein, 204 aa, Vaccinia virus
MAYMNRSDLDKLKHENIFSGNIIEDAKEFVFGSRKIYTDSVDDLIELYSLAKYLNNENLKDVVIERMDYVCKYIGKDNWSTIYSFYKENGLRNSFLRQYINNNIEEICNTDQFLKLDVDSVCDILDNDEIVVTREYTILNMVLRWLENKRVNIDDFTKVMFVIRFKFITYSELTNAIEKIAPEYRQCLQDLYHMKITRPRHFDN

SEQ ID NO: 14 - DNA, 615 bp, Vaccinia virus
  source 1..615: mol_type = unassigned DNA
atggcgtatatgaacagatcagatctcgataaactaaaacacgaaaatatcttttctgggaatattatagaggacgccaaagagttcgtctttggatctagaaaaatatatactgattctgttgatgatctaatagaattatatagtttagccaaatatcttaacaacgaaaatcttaaagatgtagtgattgagagaatggattatgtgtgcaaatatatcggtaaagataattggagtactatatattcgttttataaagaaaatggtctacgtaatagttttctacgacaatacatcaacaataatatagaagagatatgtaacacagaccaatttctaaaattggatgtagattcagtatgtgatattctagacaacgatgagatcgtagtaactagagaatatactatcttaaacatggtattacgatggctagaaaataaaagagttaatatagacgactttactaaagttatgttcgttattcgatttaaatttataacatattctgaactcactaatgcgatagagaaaatagctccagaatacagacaatgcttacaggatctataccatatgaaaattacgcgtcctagacactttgataactag

SEQ ID NO: 15 - protein, 151 aa, Vaccinia virus
MNAYNKADSFSLESDSIKDVIHDYICWLSMTDEMRPSIGNVFKAMETFKIDAVRYYDGNIYDLAKDINAMSFDSFIRSLQNISSKKDKLTVYGTMGLLSIVVDINKGCDISNIKFAAGIIILMEYIFDDTDMSHLKVALYRRIQRRDDVDR

SEQ ID NO: 16 - DNA, 456 bp, Vaccinia virus
  source 1..456: mol_type = unassigned DNA
atgaatgcgtataataaagccgattcgttttctttagagtctgattctatcaaagatgttatacacgattatatttgttggctcagtatgactgatgaaatgagaccatctatcggaaacgtctttaaagcgatggaaacgtttaagatagacgcggttagatattacgatggtaacatatacgatttagctaaagatataaatgcgatgtcattcgacagttttataagatctctacaaaatatctcttcaaagaaagataaactcactgtttatggaaccatgggactgctgtctattgtcgtagatattaacaaaggttgtgatatatccaatatcaagttcgctgccggaataatcattttaatggagtatatttttgatgacacggatatgtctcatcttaaagtagcactctatcgtagaatacagagacgtgatgatgtagatagataa

SEQ ID NO: 17 - protein, 150 aa, Vaccinia virus
MGIQHEFDIIINGDIALRNLQLHKGDNYGCKLKIISNDYKKLKFRFIIRPDWSEIDEVKGLTVFANNYAVKVNKVDDTFYYVIYEAVIHLYNKKTEILIYSDDENELFKHYYPYISLNMISKKYKVKEENYSSPYIEHPLIPYRDYESMD

SEQ ID NO: 18 - DNA, 453 bp, Vaccinia virus
  source 1..453: mol_type = unassigned DNA
atgggtatacagcacgaattcgacatcattattaatggagatatcgcgttgagaaatttacagttacataaaggggataactacggatgcaaactaaaaattatttcgaatgattacaagaaattaaagtttagattcattatacgcccagattggtcggaaatcgacgaggtcaaaggattaaccgtatttgcaaacaactatgcggtgaaagttaataaggtagatgacacgttctattacgtaatatatgaggctgtaatacatctgtataacaaaaaaacagagatattgatttattctgatgatgagaacgagctcttcaaacactattacccatacatcagtctaaatatgattagtaaaaagtataaagttaaagaagagaactactcatccccgtatatagaacatccgttaatcccgtatagagattatgagtccatggattaa

SEQ ID NO: 19 - protein, 117 aa, Vaccinia virus
MRTLLIRYILWRNDNDQTYYNDDFKKLMLLDELVDDGDVCTLIKNMRMTLSDGPLLDRLNQPVNNIEDAKRMIAISAKVARDIGERSEIRWEESFTILFRMIETYFDDLMIDLYGEK

SEQ ID NO: 20 - DNA, 354 bp, Vaccinia virus
  source 1..354: mol_type = unassigned DNA
atgaggactctacttattagatatattctttggagaaatgacaacgatcaaacctattataatgatgattttaaaaagcttatgttgttggatgaattggtagatgacggcgatgtatgtacattgattaagaacatgagaatgacgctgtccgacggtccattgctagatagattgaatcaaccagttaataatatagaagacgctaagcgaatgatcgctattagtgccaaagtggctagagacattggtgaacgttcagaaattagatgggaagagtcattcaccatactctttaggatgattgaaacatattttgatgatctaatgattgatctatatggtgaaaaataa

SEQ ID NO: 21 - protein, 175 aa, Vaccinia virus
MTSSAMDNNEPKVLEMVYDATILPEGSSMDPNIMDCINRHINMCIQRTYSSSIIAILNRFLTMNKDELNNTQCHIIKEFMTYEQMAIDHYGEYVNAILYQIRKRPNQHHTIDLFKKIKRTPYDTFKVDPVEFVKKVIGFVSILNKYKPVYSYVLYENVLYDEFKCFINYVETKYF

SEQ ID NO: 22 - DNA, 528 bp, Vaccinia virus
  source 1..528: mol_type = unassigned DNA
atgacgtcctctgcaatggataacaatgaacctaaagtactagaaatggtatatgatgctacaattttacccgaaggtagtagcatggatccaaatatcatggattgtataaacagacacatcaatatgtgtatacaacgcacctatagttctagtataattgccattttgaatagattcctaacaatgaacaaggatgaactaaacaatacacagtgtcatataattaaagaatttatgacatacgaacaaatggcgattgaccattatggagaatatgtaaacgctattctatatcaaattcgtaaaagacctaatcaacatcacaccattgatctgtttaaaaaaataaaaagaaccccgtatgacacttttaaagtggatcccgtagaattcgtaaaaaaagttatcggatttgtatctatcttgaacaaatataaaccggtttatagttacgtcctgtacgagaacgtcctgtacgatgagttcaaatgtttcattaactacgtggaaactaagtatttctaa

SEQ ID NO: 23 - protein, 472 aa, Vaccinia virus
MIFVIESKLLQIYRNRNRNINFYTTMDNIMSAEYYLSLYAKYNSKNLDVFRNMLQAIEPSGNNYHILHAYCGIKGLDERFVEELLHRGYSPNETDDDGNYPLHIASKINNNRIVAMLLTHGADPNACDKHNKTPLYYLSGTDDEVIERINLLVQYGAKINNSVDEEGCGPLLACTDPSERVFKKIMSIGFEARIVDKFGKNHIHRHLMSDNPKASTISWMMKLGISPSKPDHDGNTPLHIVCSKTVKNVDIIDLLLPSTDVNKQNKFGDSPLTLLIKTLSPAHLINKLLSTSNVITDQTVNICIFYDRDDVLEIINDKGKQYDSTDFKMAVEVGSIRCVKYLLDNDIICEDAMYYAVLSEYETMVDYLLFNHFSVDSVVNGHTCMSECVRLNNPVILSKLMLHNPTSETMYLTMKAIEKDKLDKSIIIPFIAYFVLMHPDFCKNRRYFTSYKRFVTDYVHEGVSYEVFDDYF

SEQ ID NO: 24 - DNA, 1419 bp, Vaccinia virus
  source 1..1419: mol_type = unassigned DNA
atgatatttgttatagagagtaaattgttgcaaatatacagaaatagaaatagaaatattaatttttatactacaatggacaacattatgtcggccgagtattatctatctctttatgccaaatataatagtaaaaatttagatgtatttaggaatatgctacaagctatcgaaccttctggaaataattatcacattctacatgcgtattgtggaattaaaggactagatgaacgatttgtcgaagaacttcttcatagaggatactctccaaatgagacggatgatgatggaaattatccattgcatatagcttctaaaattaataataatagaatagtcgcgatgctgctgacgcacggcgcagatccaaacgcgtgtgataaacataataaaacacctctatattatctctcgggaacagatgatgaagtcatagagagaataaatttattggtacagtatggagccaagattaacaactcggttgatgaagaaggatgtggtccgttgttggcgtgtacagatccttcagaaagagtgtttaaaaaaataatgtccatcggattcgaagccaggatagtggataaatttggcaaaaatcatattcatagacatcttatgtcagacaatccaaaagcttctacaatctcatggatgatgaaactaggaattagtccctcaaaaccagatcatgatggaaatacacctctccatattgtatgctctaaaacagtcaagaatgtagacattatagatcttttacttccatcaacggatgttaataaacaaaacaaattcggagatagtcctcttacacttcttattaagacattgagcccagcgcatcttattaacaaattgctatcgactagcaatgttattacggatcaaacagttaatatttgtatcttttatgatagagatgatgttctagaaattattaatgataaaggaaagcaatatgattctaccgattttaagatggctgttgaagtgggatccataagatgcgtcaaatatctattagacaatgatataatttgtgaagatgctatgtactacgctgtactatctgaatacgaaacaatggtagactatctattgttcaatcattttagtgtagactctgtagttaacggtcatacatgtatgagcgaatgtgtaagactaaataacccagtcattttatcgaagctgatgttacataatcctacttctgagaccatgtatctaactatgaaagctatagaaaaagataaactagataaatctattattattccgtttatcgcgtactttgtacttatgcatccggacttttgtaaaaatcgtagatactttacttcatataaacgttttgtaactgattatgttcatgaaggagtatcttacgaagtattcgatgattatttttaa

SEQ ID NO: 25 - protein, 220 aa, Vaccinia virus
MVYKLVLLFCIASLGYSVEYKNTICPPRQDYRYWYFAAELTIGVNYDINSTIIGECHMSESYIDRNANIVLTGYGLEINMTIMDTDQRFVAAAEGVGKDNKLSVLLFTTQRLDKVHHNISVTITCMEMNCGTTKYDSDLPESIHKSSSCDITINGSCVTCVNLETDPTKINPHYLHPKDKYLYHNSEYGMRGSYGVTFIDELNQCLLDIKELSYDICYRE

SEQ ID NO: 26 - DNA, 663 bp, Vaccinia virus
  source 1..663: mol_type = unassigned DNA
atggtttacaaattagtcttgctcttttgtatcgcatctctcgggtattcggtagaatacaagaatactatatgtcctcctcgacaagattaccggtattggtactttgccgccgaactcactattggtgtaaattacgacattaattctactattattggtgagtgtcatatgagtgaaagctatatcgacagaaatgctaacatagtgttgactggatacggactagaaataaacatgaccatcatggatacggatcagagatttgtggcagcggctgagggtgttggtaaagataataaactatctgttctgttgtttaccactcagcggctagataaagttcatcataatattagtgtgacaataacatgtatggaaatgaattgtggaaccacaaaatacgatagcgatcttccggaatcaattcataaatcatcatcgtgtgatataactataaatggatcatgtgtgacatgtgttaacttagagactgatccaacaaagattaatccccattacctacaccccaaggataaatatctttatcataattctgagtatggcatgcgtggtagttatggcgtgacatttatagatgaactaaaccagtgccttcttgacataaaagaactaagttatgatatttgttatagagagtaa

SEQ ID NO: 27 - protein, 284 aa, Vaccinia virus
MDLSRINTWKSKQLKSFLSSKDTFKADVHGHSALYYAIADNNVRLVCTLLNAGALKNLLENEFPLHQAATLEDTKIVKILLFSGMDDSQFDDKGNTALYYAVDSGNMQTVKLFVKKNWRLMFYGKTGWKTSFYHAVMLNDVSIVSYFLSEIPSTFDLAILLSCIHTTIKNGHVDMMILLLDYMTSTNTNNSLLFIPDIKLAIDNKDIEMLQALFKYDINIYSVNLENVLLDDAEITKMIIEKHVEYKSDSYTKDLDIVKNNKLDEIISKNKELRLMYVNCVKKN

SEQ ID NO: 28 - DNA, 855 bp, Vaccinia virus
  source 1..855: mol_type = unassigned DNA
atggatctgtcacgaattaatacttggaagtctaagcagctgaaaagctttctctctagtaaagatacatttaaggcggatgtccatggacatagtgccttgtattatgcaatagctgataataacgtgcgtctagtatgtacgttgttgaacgctggagcattgaaaaatcttctagagaatgaatttccattacatcaggcagccacattagaagataccaaaatagtaaagattttgctattcagtggaatggatgattcacaatttgatgacaaaggaaacaccgcattgtattatgcggttgatagtggtaacatgcaaacggtgaaactgtttgttaagaaaaattggagactgatgttctatgggaaaactggatggaaaacttcattttatcatgccgtcatgcttaatgatgtaagtattgtatcatactttctttcagaaataccatctacttttgatctggctattctccttagttgtattcacaccactataaaaaatggacacgtggatatgatgattctcttgctcgactatatgacgtcgacaaacaccaataattcccttctcttcattccggacattaaattggctatagataataaagacattgagatgttacaggctctgttcaaatacgacattaatatctactctgttaatctggaaaatgtactattggatgatgccgaaataactaagatgattatagaaaagcatgttgaatacaagtctgactcctatacaaaagatctcgatatcgtcaagaataataaattggatgaaataattagcaaaaacaaggaactcagactcatgtacgtcaattgtgtaaagaaaaactaa

SEQ ID NO: 29 - protein, 259 aa, Ranavirus
MAHNRFYSEILPKQGDVTMCRVLSQSDSWDEGVYVSMMEYGNVEGYVAIGVENHRDIRKRFRKLAPGAEMCMTVLRVDREKGYVDLDDRAVNANQAYECCSRYQLRRTEMAVAERAAEYAGVKGSAVYDFLDETVRALIPGSLMSGTKGLKISSDLKQLLKEFGAEVGLDRAGRAEAVVRVPGAFFGHVLRGVTNAYDAMKEMKPDSGVNVAVYPPERGVVAVTVMAGDSEAAYWGLHAVLFKVREVVKAAGGGLCPFV

SEQ ID NO: 30 - DNA, 777 bp, Ranavirus
  source 1..777: mol_type = unassigned DNA
atggcacacaacaggttttacagcgagatactcccaaaacaaggagacgttacaatgtgcagagtcttgtctcagtcagactcttgggacgaaggcgtttacgtgtctatgatggagtacggaaacgtagagggttacgtggcaatcggcgttgaaaaccacagggacatcagaaagaggttcaggaaacttgccccaggcgctgaaatgtgcatgacggtcctcagggtagaccgcgaaaagggctacgtggatttggacgacagggccgtgaatgccaatcaggcgtacgagtgttgctccaggtaccagctgaggaggacggagatggcggtcgcagagagggcggcagagtacgctggagtaaagggatctgcagtctatgactttttggacgaaaccgtgagggccctgatacccggctccctgatgtctggaacgaaaggtctaaaaatctcttccgacctgaagcagctcctaaaagagtttggagcagaggtcggccttgacagagccggtcgggccgaggctgtcgtgagagttcccggcgccttctttgggcacgtcctgaggggtgtgacgaatgcctacgacgcaatgaaggagatgaagccagactctggggtgaatgtggccgtttaccctcccgaacgcggggttgtggctgtgactgtgatggccggcgactctgaggcggcgtactggggactgcacgccgtgctgtttaaggtgcgagaggtcgtaaaggcggccgggggaggactgtgcccctttgtg

SEQ ID NO: 31 - protein, 1273 aa, Coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 32 - DNA, 3822 bp, Coronavirus
  source 1..3822: mol_type = unassigned DNA
atgtttgtgtttcttgttttattaccattagtctctagtcagtgtgttaatttaacaaccagaactcaattaccccctgcatacactaattctttcacaagaggtgtttattaccctgacaaagttttcagatcctcagttttacattcaactcaggacttattcttacctttcttttccaatgttacttggttccatgctatacatgtctctgggaccaatggtactaagaggtttgataaccctgtcttaccatttaatgatggtgtttattttgcttccactgagaagtctaacataataagaggctggatttttggtactactttagattcgaagacccagtccttattaattgttaataacgctactaatgttgttattaaagtctgtgaatttcaattttgtaatgatccatttttaggtgtttattaccacaaaaacaacaaaagttggatggaaagtgagttcagagtttattctagtgcgaataattgcacttttgaatatgtctctcagccttttttaatggaccttgaaggaaaacagggtaatttcaaaaatttaagagaatttgtgtttaagaatattgatggttattttaaaatatattctaagcacacgcctattaatttagtgagagatctccctcagggtttttcggctttagaaccattggtagatttaccaataggtattaacatcactagatttcaaactttattagctttacatagaagttatttaactcctggtgattcttcttcaggttggacagctggtgctgcagcttattatgtgggttatttacaacctagaactttcttattaaaatataatgaaaatggaaccattacagatgctgtagactgtgcattagaccctttatcagaaacaaagtgtacgttaaaatccttcactgtagaaaaaggaatctatcaaacttctaactttagagtccaaccaacagaatctattgttagatttcctaatattacaaacttatgcccttttggtgaagtttttaacgccaccagatttgcatctgtttatgcttggaacagaaagagaatcagcaactgtgttgctgattattctgtcttatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatttagattctaaggttggtggtaattataattacttatatagattatttagaaagtctaatttaaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtattatcttttgaacttttacatgcaccagcaactgtttgtggacctaaaaagtctactaatttagttaaaaacaaatgtgtcaatttcaacttcaatggtttaacaggcacaggtgttttaactgagtctaacaaaaagtttctgcctttccaacaatttggcagagacattgctgacactactgatgctgtcagagatccacagacattagagattttagacattacaccatgttcttttggtggtgtcagtgttataacaccaggaacaaatacttctaaccaggttgctgttttatatcaggatgttaactgcacagaagtccctgttgctattcatgcagatcaattaactcctacttggagagtttattctacaggttctaatgtttttcaaacaagagcaggctgtttaataggggctgaacatgtcaacaactcatatgagtgtgacatacccattggtgcaggtatatgcgctagttatcagactcagactaattctcctgggagcgcaagtagtgtagctagtcaatccatcattgcctacactatgtcattaggtgcagaaaattcagttgcttactctaataactctattgccatacccacaaattttactattagtgttaccacagaaattttaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagagatcaccaattgaagatttacttttcaacaaagtgacattagcagatgctggcttcatcaaacaatatggtgattgcttaggtgatattgctgctagagacttaatttgtgcacaaaagtttaacggcttaactgttttaccacctttattaacagatgaaatgattgctcaatacacttctgcattattagcgggtacaatcacttctggttggacctttggtgcaggtccggcattacaaataccatttccgatgcaaatggcttatagatttaatggtattggagttacacagaatgttttatatgagaaccaaaaattaattgccaaccaatttaatagtgctattggcaaaattcaagactcattatcttccacaccaagtgcattaggaaaattacaagatgtggtcaaccaaaatgcacaagctttaaacacgttagttaaacaattaagctccaattttggtgcaatttcaagtgttttaaatgatatcttatcaagattagacccaccagaggctgaagtgcaaattgatagattaatcacaggcagattacaaagtttacagacatatgtgactcaacaattaattagagctgcagaaatcagagcttctgctaatttagctgctactaaaatgtcagagtgtgtattaggacaatcaaaaagagttgatttctgtggaaagggctatcatttaatgtccttccctcagtcagcacctcatggtgtagtcttcttacatgtgacttatgtccctgcacaagaaaagaacttcacaactgctcctgccatttgtcatgatggaaaagcacactttcctagagaaggtgtctttgtttcaaatggcacacactggtttgtaacacaaagaaatttctatgaaccacaaatcattactacagacaacacatttgtgtctggtaactgtgatgttgtaataggaattgtcaacaacacagtttatgatcctttacaacctgaattagactcattcaaggaggagttagataaatattttaagaatcatacatcaccagatgttgatttaggtgacatctctggcattaatgcttcagttgtaaacattcaaaaagaaattgacagattaaatgaggttgccaagaatttaaatgaatctttaatcgatttacaagaattaggaaagtatgagcagtatataaaatggccatggtacatttggttaggttttatagctggcttaattgccatagtaatggtgacaattatgttatgctgtatgaccagttgctgtagttgtttaaagggctgttgttcttgtggatcctgctgcaaatttgatgaagacgactctgagccagtgttaaaaggagtcaaattacattacacataa

(19) United States
(12) Patent Application Publication
(10) Pub. No.: US 2026/0218234 A1
(43) Pub. Date: Jul. 30, 2026

(54) MODIFIED IMMUNIZATION VECTORS

(71) Applicant: ARIZONA BOARD OF REGENTS ON BEHALF OF ARIZONA STATE UNIVERSITY, Scottsdale, AZ (US)

(72) Inventors: Bertram Jacobs, Tempe, AZ (US); Karen Kibler, Scottsdale, AZ (US)

(21) Appl. No.: 19/157,913

(22) PCT Filed: Aug. 22, 2024
(86) PCT No.: PCT/US24/43384

§ 371 Date: Aug. 19, 2025

Related U.S. Application Data
(60) Provisional application No. 63/533,971, filed on Aug. 22, 2023.

(51) Int. Cl. C12N 15/86; A61K 39/00; A61K 39/215; A61P 37/04; C07K 14/005

(52) U.S. Cl. CPC C12N 15/86; A61K 39/215; A61P 37/04; C07K 14/005; A61K 2039/5256; C12N 2710/24122; C12N 2710/24143; C12N 2770/20022; C12N 2770/20034

(57) ABSTRACT

The disclosure relates to recombinant modified NYVAC vectors encoding one or more virus immunogens (or antigens), preferably derived from a coronavirus or an influenza virus, for use in immunological compositions, as well as methods for making and using the same.

### RELATED APPLICATIONS

This application claims priority to Ser. No. 63/533,971 filed on Aug. 22, 2023, which is incorporated by reference herein in its entirety.

### FIELD OF THE DISCLOSURE

The disclosure relates to modified NYVAC vectors encoding one or more viral immunogens, preferably one or more influenza or coronavirus immunogens (or antigens) for use in immunological compositions, as well as methods for making and using the same.

### SEQUENCE LISTING

This application contains a Sequence Listing which has been submitted electronically in ASCII format via EFS-Web and hereby incorporated by reference in its entirety. Said ASCII copy, created on 22 Aug. 2023, is named M23-175L-PR-1-f_ST25.TXT and is 67 kilobytes in size.

### BACKGROUND OF THE DISCLOSURE

Coronavirus disease 2019 (COVID-19) is caused by the novel coronavirus named SARS-CoV-2 by the Coronavirus Study Group (a working group of the International Committee on Taxonomy of Viruses) based on phylogeny, taxonomy and established practice (BioRxiv; doi.org/10.1101/2020.02.07.937862)). It is known as a new strain that was not been previously identified in humans and was first reported in Wuhan, Hubei Province, China. Genomic sequencing has shown that it is a positive-sense, single-stranded RNA coronavirus (GenBank Accession No. MN908947.3; RefSeq NC_045512; “Wuhan seafood market pneumonia virus isolate Wuhan-Hu-1”).

Influenza is known to be caused by the influenza virus. There are around a billion cases of seasonal influenza annually, including 3-5 million cases of severe illness. It causes 290 000 to 650 000 respiratory deaths annually. Ninety-nine percent of deaths in children under 5 years of age with influenza-related lower respiratory tract infections are in developing countries. Symptoms begin 1-4 days after infection and usually last around a week. Influenza spreads easily between people when they cough or sneeze. Vaccination is the best way to prevent the disease.

Immunogenic compositions (e.g., vaccines) have been produced to prevent and/or treat viral infections by, e.g., coronavirus and influenza, but have not been shown to be completely effective, or perhaps to be without side effects. There is need in the art for effective immunological compositions and methods for immunizing animals and humans using recombinant vectors. It is known in the art that certain vectors (e.g., replication-incompetent vaccinia vectors) are insufficient as immunomodulators. As described herein, modification of such vectors provides a solution to these problems.

### BRIEF DESCRIPTION OF THE DRAWINGS

FIG. 1. Construction of NYVAC-KC-ΔB8R-ΔB19R vector.

FIG. 2. Construction of NYVAC-KC-ΔB8R-ΔB19R-psfSpike vector.

FIG. 3. Illustration of NYVAC-KC-ΔB8RΔB19R DNA.

FIG. 4. Clinical scores for NYVAC-KC-psfSpike vector and NYVAC-KC-ΔB8R-ΔB19R-psfSpike vector (NYVAC-KC-ΔΔ-psfSpike) using mouse model.

FIG. 5. Weight of mice administered NYVAC-KC-psfSpike vector or NYVAC-KC-ΔB8R-ΔB19R-psfSpike vector (NYVAC-KC-AA-psfSpike).

FIG. 6. Weight of mock-vaccinated mice.

### SUMMARY OF THE DISCLOSURE

Disclosed herein are compositions and reagents for immunizing human beings against coronavirus immunogens (or antigens) by inducing or enhancing the immune response against the coronavirus or influenza virus. In preferred embodiments, the recombinant vector is a modified NYVAC vector lacking the non-essential region of the vaccinia virus genome containing C1L (SEQ ID Nos. 5-6), C2L (SEQ ID Nos. 7-8), C3L (SEQ ID Nos. 9-10), C4L (SEQ ID Nos. 11-12), C5L (SEQ ID Nos. 13-14), C6L (SEQ ID Nos. 15-16), NIL (SEQ ID Nos. 19-20), N2L (SEQ ID Nos. 21-22), M1L (SEQ ID Nos. 23-24), and M2L (SEQ ID Nos. 25-26); is unable to express B8R (SEQ ID NO: 2) and B19 (SEQ ID NO: 14); and, encodes C7L (SEQ ID Nos. 17-18) and K1L (SEQ ID Nos. 27-28) as well as at least one coronavirus immunogen (or antigen). In preferred embodiments, the coronavirus immunogen is a coronavirus spike protein (preferably the psfSpike polypeptide of SEQ ID NO: 31 preferably encoded by SEQ ID NO: 32). In preferred embodiments, the one or more antigens are derived from the influenza trimeric hemagglutinin (HA) glycoprotein neuraminidase, nucleoprotein, or extracellular domain of matrix 2 ion channel protein (M2). Compositions comprising such vectors and methods for using such compostions are also provided. Methods for constructing and using such vectors are described herein.

### DETAILED DESCRIPTION

The present disclosure provides compositions and methodologies useful for expressing nucleic acids and the polypeptides, peptides, or nucleic acids encoded thereby using recombinant vectors. In one embodiment, the compositions comprise recombinant vectors for introducing or altering the expression of a polypeptide, peptide, or nucleic acid in a host. In some embodiments, the compositions may include one or more recombinant viruses comprising polynucleotides encoding polypeptides, peptides, or polynucleotides that were not previously expressed by the virus or are normally expressed in different amounts or at different times in the life cycle of the virus. In certain embodiments, polynucleotides can be incorporated into and/or modified (e.g., inactivated by deletion or other modification) viral genome to produce a recombinant modified virus with altered characteristics as compared to the non-modified virus. In some embodiments, the incorporated and/or modified polynucleotides encode polypeptides, peptides, or polynucleotides that alter the growth characteristics, infectivity, host range, replicative capacity, or immunogenicity of the recombinant virus as compared to the non-modified virus. Such polynucleotides may be introduced and/or modified alone or in combination with other polynucleotides, such as those described below (e.g., encoding one or more immunogens).

In some embodiments, the recombinant viral vectors may be modified by deleting polynucleotides (e.g., a gene) normally found within the vector therefrom, and/or reducing the activity or expression of the protein encoded by the viral genome, and/or otherwise modifying the viral genome. For instance, the poxvirus NYVAC (described in more detail below) was derived from the Copenhagen vaccinia strain using transient dominant selection (Falkner & Moss, 1990) which allows for deletion of one or more target genes without incorporation of a polynucleotide encoding a selectable marker into the viral genome. In some embodiments, one or more polynucleotides within the viral genome may be completely or partially deleted and/or inactivated with or without partial deletion (e.g., such the polypeptide encoded thereby is not expressed and/or is not functional in a cell). In some embodiments, partial deletion may be accomplished by removing a portion of a polynucleotide encoding a polypeptide from the genome of the viral vector (“vector genome”). As referred to herein, the vector genome may refer to the polynucleotide encoding the various factors required for the viability of a replication-competent or replication-incompetent viral vector, the polynucleotide making up a non-viral (e.g., bacterial, eukaryotic) or viral plasmid vector, or the like.

The preferred vector for use as disclosed herein is a modified version of vaccinia, NYVAC (vP866), which was derived from the Copenhagen vaccine strain of vaccinia virus by deleting six nonessential regions of the genome encoding known or potential virulence factors (see, for example, U.S. Pat. Nos. 5,364,773 and 5,494,807; Tartaglia, et al. Virology, 188:217-232 (1992)). The deletion loci were also engineered as recipient loci for the insertion of foreign genes. The deleted regions of the parental NYVAC vector are the thymidine kinase gene (TK; J2R); hemorrhagic region (u; B13R+B14R); A type inclusion body region (ATI; A26L); hemagglutinin gene (HA; A56R); host range gene region (C7L-K1L); and, large subunit, ribonucleotide reductase (14L). NYVAC (vP866), vP994, vCP205, vCP1433, placZH6H4Lreverse, pMPC6H6K3E3 and pC3H6FHVB were also deposited with the ATCC under the terms of the Budapest Treaty, accession numbers VR-2559, VR-2558, VR-2557, VR-2556, ATCC-97913, ATCC-97912, and ATCC-97914, respectively. These modified vaccinia vectors were shown to exhibit altered host range and to be useful for expressing immunogens within a wide range of species. Such NYVAC vectors have been shown to be useful for expressing immunogens (antigens) (see, for example, U.S. Pat. Nos. 6,265,189 and 9,670,506) and used as recombinant vaccines against numerous pathogens and tumours in animal models and humans (Myagkikh et al., 1996; Benson et al., 1998; Siemens et al., 2003; Franchini et al., 2004). Clinical trials using NYVAC-based vectors showed an acceptable safety profile, with induction of high levels of immunity against heterologous antigens (Kanesa-thasan et al., 2000; Gómez, C. E et al. 2007; Harari, A et al, 2008). Such vectors may be further modified by insertion or deletion of additional polynucleotides using the techniques described herein. Suitable polynucleotides may include, for example, those involved in host range, apoptosis, signaling, cytokine and/or chemokine expression or activity, cytokine and/or chemokine pathways, and/or the like, resulting in novel biological characteristics of the vectors.

The nomenclature of these sequences is related to the Copenhagen strain of vaccinia virus (GenBank Accession No. M35027; Goebel, et al. The complete DNA sequence of vaccinia virus. Virology 179 (1), 247-266 (1990); Goebel, et al. Appendix to ‘The complete DNA sequence of Vaccinia virus’. Virology 179, 517-563 (1990)). Any of such polynucleotides may be modified (e.g, incorporated into a recombinant vector or as part of a composition containing multiple recombinant vectors) in combination with any other of such polynucleotides. Other polynucleotides may also be suitable for modification in vaccinia or in other viruses (e.g., MVA, avipox, and the like).

In some embodiments, polynucleotides encoding immunomodulatory polypeptides are selectively deleted from a vector genome. Polynucleotides encoding immunogens may also be incorporated into the vector genome. This may lead to modulation of virus-host cell interactions and “improvement” in the immunological profiles of the modified vectors as candidate vaccines. By “improvement” is meant that an immune response against a target antigen is induced or enhanced. In certain embodiments, the modified vectors may exhibit improved safety profiles as compared to non-modified (e.g., parental) vectors.

Polynucleotides suitable for modification (e.g., deletion from, alteration of sequence, or incorporation into a vector genome) may include, for example, any polynucleotide that provides a desired effect on a virus (e.g., replication) or the host (e.g., an improved immune response against an antigen). For instance, within the NYVAC vector (preferably modified NYVAC as exemplified herein), one or more candidate polynucleotides for modification may include polynucleotides that may be characterized as immunomodulators, and those affecting viral host range, one or more signalling pathways, apoptosis, secreted proteins (e.g., those binding host cytokines and/or chemokines).

The B8R gene (open reading frame (“ORF”) shown in SEQ ID NO. 1) encodes the B8R protein (SEQ ID NO. 2) with amino acid similarity to the extracellular domain of the IFN-γ receptor (Alcami & Smith, 1995; Mossman et al., 1995). The protein B8 binds and inhibits IFN-γ from a wide variety of species but not the mouse. Deletion of B8R from WR did not alter virus replication or virulence in mouse models (Symons, et al. 2002a).

The B19R gene of VACV (ORF shown in SEQ ID NO. 3 encoding the B19R polypeptide, SEQ ID NO. 4) is equivalent to the B18R gene of VACV WR and encodes a type I IFN (α,β)-receptor homolog. Protein B19 binds and inhibits type I IFN from a wide variety of species except murine IFN which binds but does not inhibit it. Deletion of B19R from VACV WR has been shown to cause attenuation in a murine intranasal model.

Exemplary polynucleotides that are candidates for modification, most preferably deletion (or at least inactivation of the polypeptide encoded thereby) in preferred modified NYVAC vectors of this disclosure, include those encoding, for example, B8R (SEQ ID NO. 1 (polypeptide), SEQ ID NO. 2 (preferred polynucleotide encoding SEQ ID NO. 1)) and/or B19R (SEQ ID NO. 3 (polypeptide), SEQ ID NO: 4 (preferred polynucleotide encoding SEQ ID NO. 3)). In preferred embodiments, the modified NYVAC vector cannot express either of B8R or B19R, preferably due to modification (preferably deletion) of the coding sequences thereof from the NYVAC genome to provide a preferred modified NYVAC. In certain embodiments, suitable and exemplary polynucleotides may encode immunomodulatory polypeptides that interact with, for example, one or more interferons, cytokines and/or chemokines.

Within vaccinia, the C1L (SEQ ID NO. 5), C2L (SEQ ID NO. 7), C3L (SEQ ID NO. 9), C4L (SEQ ID NO. 11), C5L (SEQ ID NO. 13), C6L (SEQ ID NO. 15), C7L (SEQ ID NO. 17), NIL (SEQ ID NO. 19), N2L (SEQ ID NO. 21), M1L (SEQ ID NO. 23), M2L (SEQ ID NO. 25) and K1L (SEQ ID NO. 27) polypeptides have been shown to be involved in defining the “host range” or replication competence of the virus. Polynucleotides encoding such host range polypeptides are illustrated in SEQ ID NOS. 6, 8, 10, 12, 14, 16, 18, 20, 22, 24, 26, and 28. In the NYVAC virus, these genes have been deleted. In certain embodiments, one or more polynucleotides representing one or more of these host range genes may be introduced into the genome of a viral vector to affect the replication competence of the vector. In NYVAC, for example, one or more polynucleotides representing one or more of such host range genics may be re-incorporated into the NYVAC genome to modify its replication competence. In certain embodiments, as shown in the Examples, polynucleotides encoding C7L (e.g., SEQ ID NOS. 17, 18) and K1L (e.g., SEQ ID NOS. 27, 28) were shown to effect replication competence of NYVAC. In certain embodiments, the modified NYVAC vector expresses at least one, two, three, four, five, six, seven, eight, nine, ten, eleven or twelve of C1L (e.g., SEQ ID NOS. 5, 6), C2L (e.g., SEQ ID NOS. 7, 8), C3L (e.g., SEQ ID NOS. 9, 10), C4L (SEQ ID NOS. 11, 12), C5L (e.g., SEQ ID NOS. 13, 14), C6L (e.g., SEQ ID NOS. 15, 16), C7L (e.g., SEQ ID NOS. 17, 18), NIL (SEQ ID NOS. 19, 20), N2L (e.g., SEQ ID NOS. 21, 22), M1L (e.g., SEQ ID NOS. 23, 24), M2L (e.g., SEQ ID NOS. 25, 26), and K1L (e.g., SEQ ID NOS. 27, 28). Various combinations of such polynucleotides and/or polypeptides, as would be apparent to one of skill in the art, may be utilized in vectors. Certain of these polynucleotides (preferably C7L (e.g., SEQ ID NOS. 17, 18) and K1L (e.g., SEQ ID NOS. 27, 28)) and/or polypeptides may also be incorporated into modified NYVAC vectors modified not to express B8R (SEQ ID NOS. 1, 2) and/or B19R (SEQ ID NOS. 3, 4) (the NYVAC-KC-ΔB8R-ΔB19R vector described and used in the Examples herein). Thus, the modified NYVAC vectors of this disclosure preferably express neither B8R or B19R. Suitable recombinant vectors for introduction or re-introduction of such host range genes include those from which such sequences have been previously deleted or those that otherwise do not contain such genes within the vector genome. It is also possible to modify such host range genes such that their function is altered by, for example, altering the timing or character (e.g., expression level) of expression within a host cell.

Polynucleotides encoding other polypeptides, peptides, or nucleic acids affecting the activity of a recombinant vector (e.g., recombinant virus) may also be incorporated into the vector. In certain embodiments, polynucleotides representing genes from other organisms (exogenous genes) may be incorporated into the vector. The polynucleotides may be inserted into a polynucleotide by insertion, either de novo or by replacement of an existing polynucleotide sequence within the vector genome. For instance, a polynucleotide may replace a gene of a virus. For example, the ranavirus eIF2α-like gene (“eIF2αH”) from Ambystoma tigrinum virus isolate YEL protein gene (GenBank Accession No. EU512333; version EU512333.1; GI: 170180537; “ATV eIF2αH”; SEQ ID NO. 29 encoded by SEQ ID NO. 30; see, e.g., U.S. Pat. No. 7,431,929) may be utilized. ATV eIF2αH encodes a potent, non-dsRNA-binding inhibitor of RNA-dependent protein kinase (PKR). In one embodiment, a polynucleotide encoding ATV eIF2αH (e.g., SEQ ID NO. 30) may be incorporated into a modified NYVAC vector described herein. Without being limited to any particular theory of operation, it is believed that ATV eIF2QH induces signal transduction through NF-κB and IRF-3, while sparing viral protein synthesis from the inhibitory effects of PKR activation. In certain embodiments, a recombinant virus may be produced that exhibits little, decreased, or no replication competence but also induces an immune response in a host. Such a virus may provide an optimal recombinant vector that represents a “compromise” between replication competent that may cause complications in hosts, and replication deficient recombinant vectors that may fail to induce an immune response or may induce a sub-optimal immune response.

In certain embodiments, the recombinant NYVAC vector may include a polynucleotide encoding ATV eIF2αH (e.g., SEQ ID NOS. 29, 30). For instance, a modified NYVAC vector of this disclosure can be one in which the E3L gene was deleted and replaced by a polynucleotide encoding ATV eIF2αH (SEQ ID NO. 30 encoding SEQ ID NO. 29; see, e.g., U.S. Pat. No. 7,431,929). It has been observed that this modified virus induces host cell production of IFN, exhibits increased sensitivity to IFN, and induces a potent Th1-dominated immune response at low doses. Other embodiments, as could be derived from this disclosure, may also be suitable for use.

In some embodiments, the compositions include one or more recombinant vectors (preferably a modified NYVAC vector) encoding one or more immunogens that may be used to induce or enhance an anti-viral immune response that is either prevents infection by, ameliorates the symptoms of, and/or reduces the spread of viral infection. In preferred embodiments, the immunogens are derived from and induce and an immune response against infection by a virus infectious to a mammal, preferably where the mammal is a human being. In preferred embodiments, the virus in an influenza virus or a coronavirus.

Exemplary common coronaviruses include 229E, NL63, HKU1, and OC43, which cause mild to moderate upper respiratory symptoms that can include cough, fever, headache, runny nose, and sore throat. Antigens from any one or more of such coronaviruses, or any other one or more common coronaviruses, can be encoded by the one or more recombinant vectors (preferably a modified NYVAC vector) of this disclosure. In preferred embodiments, the coronavirus is a SARS-CoV-1 or SARS-CoV-2 virus (Betacoronavirus pandemicum). The SARS-CoV-2 coronavirus strain causes COVID-19 (the virus also being terms 2019-nCOV, HCoV-19, and hCoV-19). SARS-CoV-2 variants include alpha, bets, gamma, delta, omicron, epsilon, zeta, eta, theta, iota, kappa, lambda, mu, cluster 5, lineage B.1.617, among others as the list is evolving. In preferred embodiments, one or more coronavirus proteins including but not limited to the coronavirus spike protein (e.g., SEQ ID NO: 31 (psfSpike polypeptide of the SARS-CoV-2 Washington strain) encoded by SEQ ID NO: 32) and/or nucleoprotein. These are other antigens/immunogens from any such coronaviruses can be encoded by and expressed from the one or more recombinant vectors (preferably a modified NYVAC vector) of this disclosure.

Exemplary influenza viruses from which the one or more antigens (or immunogens) can be derived include but are not limited to Influenza A, Influenza B, Influenza C, or Influenza D (to the extent such a virus can infect and/or cause illness in humans). Subtypes, clades and subclades of influenza viruses are known to those of skill in the art. For instance, exemplary Influenza A viruses can express various combinations of 18 different hemagglutinin subtypes and 11 different neuraminidase subtypes (H1 through H18 and N1 through N11 (e.g., subtypes A (H1N1 (e.g., 2009 H1N1), H3N2)). Exemplary Influenza B viruses include those of the Victoria (e.g., VIA, V1A.1, V1A.2, V1A.3) and Yamagata lineages (e.g., Y1, Y2, or Y3 types). Antigens (or immunogens) from any such influenza virus, or any others that may develop over time, can be used as disclosed herein. In preferred embodiments, the one or more antigens are derived from the influenza trimeric hemagglutinin (HA) glycoprotein (e.g., (A/Thailand/1 (KAN-1)/2004, GenBank AY555150)), neuraminidase, nucleoprotein (NP) (NP (A/Thailand/1 (KAN-1)/2004, GenBank AAV35112 and A/PR/8/34, GenBank AAM75159)), extracellular domain of matrix 2 ion channel protein (M2) ((A/Thailand/1 (KAN-1)/2004, GenBank AAV35111)) (see, e.g., Rao, et al. PLOS ONE, 5(3): e9812 (2010)). In preferred embodiments, the influenza proteins are expressed as glycoproteins with the relevant (i.e., capable of inducing an immune response) antigen structures thereupon. In preferred embodiments, the influenza proteins induce cross-reactive immune responses (i.e., an immune response that is protective and/or therapeutic against more than one influenza strain (e.g., more than one subtype such as at least two of Influenza A H1, H2, G5, H6 and H9). Other influenza antigens are also contemplated herein as would be understood by those of ordinary skill in the art.

The immune response is preferably a protective immune response, but therapeutic immune responses (e.g., to alleviate an existing viral infection) can also result from administration of a preferred modified NYVAC expression vector to a human being (e.g., as part of a pharmaceutical composition). A protective immune response is one that prevents and/or reduces the ability of an influenza or coronavirus to infect and/or effectively replicate in a human being, and/or be transmitted to another human being. A therapeutic immune response could also be protective (thus, a composition could be both protective and therapeutic), but if administered during an active viral infection can alleviate or reduce the time during which symptoms of viral infection is experienced by the human being (e.g., fever, cough, headache, muscle/joint pain, malaise, sore throat, nasal congestion). The immune response is preferably induced in a human being of any age, including infants and the elderly.

The construction and characteristics of a preferred modified NYVAC expression vector is described in U.S. Pat. No. 9,670,506 filed on Apr. 10, 2010 (Pantaleo, et al.) As shown in U.S. Pat. No. 9,670,506, deletion of the B8R (SEQ ID Nos. 1-2) and B19 (SEQ ID Nos. 13-14) from a modified NYVAC vector was shown to provide improved immunogenicity characteristics to the vector. To engineer that modified NYVAC, the parental NYVAC vector was modified by deleting a non-essential region of the vaccinia virus genome containing C1L (SEQ ID Nos. 5-6), C2L (SEQ ID Nos. 7-8), C3L (SEQ ID Nos. 9-10), C4L (SEQ ID Nos. 11-12), C5L (SEQ ID Nos. 13-14), C6L (SEQ ID Nos. 15-16), C7L (SEQ ID Nos. 17-18), NIL (SEQ ID Nos. 19-20), N2L (SEQ ID Nos. 21-22), M1L (SEQ ID Nos. 23-24), M2L (SEQ ID Nos. 25-26), and K1L (SEQ ID Nos. 27-28) (see Tartaglia, et al. Virology, 188:217-232 (1992)). Deletion of genes in this region rendered NYVAC replication-defective in human cells. A second modified NYVAC vector was produced that includes C7L and K1L inserted back into the region of the NYVAC genome from which C1L, C2L, C3L, C4L, C5L, C6L, NIL, N2L, M1L, and M2L were deleted. As shown in U.S. Pat. No. 9,670,506, replication competence of NYVAC was restored by re-insertion of these two host range genes C7L and K1L into NYVAC to produce NYVAC-KC. B8R and B19R were deleted from NYVAC-KC to produce the modified vector NYVAC-KC-ΔB8R-ΔB19R (also referred to as NYVAC-KC-AA). Thus, the preferred modified NYVAC vector of this disclosure lacks the B8R and B19R open reading frames and includes the C7L and K1L open reading frames in the genome. As shown in U.S. Pat. No. 9,670,506, the NYVAC-KC-ΔB8R-ΔB19R vector was used to induce a potent immune response against HIV antigens by incorporating HIV antigens therein. In preferred embodiments, this disclosure relates to the use of the modified NYVAC vector NYVAC-KC-ΔB8R-ΔB19R vector to induce an immune response against the coronavirus spike protein and, preferably, the coronavirus or influenza virus per se. In the most preferred embodiments, the coronavirus immunogen encoded by the modified NYVAC vector is the coronavirus immunogen having the amino acid sequence of SEQ ID NO: 31 (psfSpike polypeptide of the SARS-CoV-2 Washington strain) that can, in preferred embodiments, be encoded by SEQ ID NO: 32. In some preferred embodiments, the immune response against mouse-adapted SARS2-N501Y_{MA30} as determined using the mouse model (see, e.g., the Examples herein) using routine techniques known to those of ordinary skill in the art. In preferred embodiments, the modified NYVAC vector encodes one or more influenza antigens derived from the influenza trimeric hemagglutinin (HA) glycoprotein (e.g., (A/Thailand/1 (KAN-1)/2004, GenBank AY555150)), neuraminidase, nucleoprotein (NP) (NP (A/Thailand/1 (KAN-1)/2004, GenBank AAV35112 and A/PR/8/34, GenBank AAM75159)), extracellular domain of matrix 2 ion channel protein (M2) ((A/Thailand/1 (KAN-1)/2004, GenBank AAV35111)) (see, e.g., Rao, et al. PLOS ONE, 5(3): e9812 (2010)).

In preferred embodiments, then, the modified NYVAC vector disclosed herein encodes at least one immunogen (preferably a coronavirus spike protein or influenza virus protein) encoded by a nucleotide sequence in expressible form within the modified NYVAC expression vector genome. An immunogen may be a moiety (e.g., polypeptide, peptide or nucleic acid) that induces or enhances the immune response of a host to whom or to which the immunogen is administered. An immune response may be induced or enhanced by either increasing or decreasing the frequency, amount, or half-life of a particular immune modulator (e.g., the expression of a cytokine, chemokine, co-stimulatory molecule). This may be directly observed within a host cell containing a polynucleotide of interest (e.g., following infection by a recombinant virus) or within a nearby cell or tissue (e.g., indirectly). The immune response is typically directed against a target antigen. For example, an immune response may result from expression of an immunogen in a host following administration of a nucleic acid vector encoding the immunogen to the host. The immune response may result in one or more of an effect (e.g., maturation, proliferation, direct- or cross-presentation of antigen, gene expression profile) on cells of either the innate or adaptive immune system. For example, the immune response may involve, effect, or be detected in innate immune cells such as, for example, dendritic cells, monocytes, macrophages, natural killer cells, and/or granulocytes (e.g., neutrophils, basophils or eosinophils). The immune response may also involve, effect, or be detected in adaptive immune cells including, for example, lymphocytes (e.g., T cells and/or B cells). The immune response may be observed by detecting such involvement or effects including, for example, the presence, absence, or altered (e.g., increased or decreased) expression or activity of one or more immunomodulators such as a hormone, cytokine, interleukin (e.g., any of IL-1 through IL-35), interferon (e.g., any of IFN-I (IFN-α, IFN-β, IFN-ε, IFN-κ, IFN-τ, IFN-ζ, IFN-ω), IFN-II (e.g., IFN-γ), IFN-III (IFN-λ1, IFN-λ2, IFN-λ3)), chemokine (e.g., any CC cytokine (e.g., any of CCL1 through CCL28), any CXC chemokine (e.g., any of CXCL1 through CXCL24), Mip1a), any C chemokine (e.g., XCL1, XCL2), any CX3C chemokine (e.g., CX3CL1)), tumor necrosis factor (e.g., TNF-α, TNF-β)), negative regulators (e.g., PD-1, IL-T) and/or any of the cellular components (e.g., kinases, lipases, nucleases, transcription-related factors (e.g., IRF-1, IRF-7, STAT-5, NFKB, STAT3, STAT1, IRF-10), and/or cell surface markers suppressed or induced by such immunomodulators) involved in the expression of such immunomodulators. The presence, absence or altered expression may be detected within cells of interest or near those cells (e.g., within a cell culture supernatant, nearby cell or tissue in vitro or in vivo, and/or in blood or plasma). Administration of the immunogen may induce (e.g., stimulate a de novo or previously undetected response), or enhance or suppress an existing response against the immunogen by, for example, causing an increased antibody response (e.g., amount of antibody, increased affinity/avidity) or an increased cellular response (e.g., increased number of activated T cells, increased affinity/avidity of T cell receptors). In certain embodiments, the immune response may be protective, meaning that the immune response may be capable of preventing initiation or continued infection of or growth within a host and/or by eliminating an agent (e.g., a causative agent, such as HIV) from the host. The Examples herein show that expression of SEQ ID NO: 31 (psfSpike polypeptide of the SARS-CoV-2 Washington strain) encoded by SEQ ID NO: 32 serves as a coronavirus immunogen using the mouse immune response against mouse-adapted SARS2-N501Y_{MA30}.

In some embodiments, derivatives of polypeptides, peptides, or polynucleotides incorporated into or expressed by the vectors described herein including, for example, fragments and/or variants thereof may be utilized. Derivatives may result from, for example, substitution, deletion, or addition of amino acids or nucleotides from or to the reference sequence (e.g., the parental sequence). A derivative of a polypeptide or protein, for example, typically refers to an amino acid sequence that is altered with respect to the referenced polypeptide or peptide. A derivative of a polypeptide typically retains at least one activity of the polypeptide. A derivative will typically share at least approximately 60%, 70%, 80%, 90%, 95%, or 99% identity to the reference sequence. With respect to polypeptides and peptides, the derivative may have “conservative” changes, wherein a substituted amino acid has similar structural or chemical properties. A derivative may also have “nonconservative” changes. Exemplary, suitable conservative amino acid substitutions may include, for example, those shown in Table 1:

TABLE 1

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

Other amino acid substitutions may be considered non-conservative. Derivatives may also include amino acid or nucleotide deletions and/or additions/insertions, or some combination of these. Guidance in determining which amino acid residues or nucleotides may be substituted, inserted, or deleted without abolishing the desired activity of the derivative may be identified using any of the methods available to one of skill in the art. Derivatives may also refer to a chemically modified polynucleotide or polypeptide. Chemical modifications of a polynucleotide may include, for example, replacement of hydrogen by an alkyl, acyl, hydroxyl, or amino group. A derivative polynucleotide may encode a polypeptide which retains at least one biological or immunological function of the natural molecule. A derivative polypeptide may be one modified by glycosylation, pegylation, biotinylation, or any similar process that retains at least one biological or immunological function of the polypeptide from which it was derived. For the purposes of this disclosure, a suitable derivative is one that induces an anti-coronavirus immune response in a host animal (e.g., as shown in the Examples).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 10, at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

As mentioned above, this disclosure relates to compositions comprising recombinant vectors, the vectors per se, and methods of using the same. A “vector” is any moiety (e.g., a virus or plasmid) used to carry, introduce, or transfer a polynucleotide or interest to another moiety (e.g., a host cell). In certain cases, an expression vector is utilized. An expression vector is a nucleic acid molecule containing a polynucleotide of interest encoding a polypeptide, peptide, or polynucleotide and also containing other polynucleotides that direct and/or control the expression of the polynucleotide of interest. Expression includes, but is not limited to, processes such as transcription, translation, and/or splicing (e.g., where introns are present). Expression vectors typically comprise one or more flanking polynucleotides “operably linked” to a heterologous polynucleotide encoding a polypeptide. As used herein, the term “operably linked” refers to a linkage between polynucleotide elements in a functional relationship such as when promoter or enhancer affects transcription of a polynucleotide of interest (e.g., a coding sequence). Flanking polynucleotides may be homologous (e.g., from the same species and/or strain as the host cell), heterologous (e.g., from a species other than the host cell species and/or strain), hybrid (e.g., a combination of flanking sequences from more than one source), or synthetic, for example. All polynucleotides referred to herein are typically incorporated into vectors in expressible form, meaning that such polynucleotides are capable of being expressed from the expression vector transformed into a cell or after incorporation of the expression vector or portions thereof into the genome of an infected or transformed cell, such that the polypeptide encoded thereby is expressed in the infected or transformed cell. The flanking sequences described herein typically assist in achieving expression in the infected or transformed cell. In certain embodiments, it is preferred that the flanking polynucleotide includes a transcriptional regulatory region that drives expression of a polynucleotide of interest in an environment such as a target cell. The transcriptional regulatory region may comprise, for example, a promoter, enhancer, silencer, repressor element, or combinations thereof. The transcriptional regulatory region may be either constitutive, tissue-specific, cell-type specific (e.g., the region is drives higher levels of transcription in a one type of tissue or cell as compared to another) and/or regulatable (e.g., responsive to interaction with a compound such as tetracycline). The source of a transcriptional regulatory region may be any prokaryotic or eukaryotic organism, any vertebrate or invertebrate organism, or any plant, provided that the flanking polynucleotide functions in an environment (e.g., a cell) by causing transcription of a polynucleotide within that environment. A wide variety of suitable transcriptional regulatory regions are available to one of skill in the art. Suitable transcriptional regulatory regions include, for example, the synthetic e/l promoter; the CMV promoter (e.g., the CMV-immediate early promoter); promoters from eukaryotic genes (e.g., the estrogen-inducible chicken ovalbumin gene, the interferon genes, the gluco-corticoid-inducible tyrosine aminotransferase gene, and the thymidine kinase gene); and the major early and late adenovirus gene promoters; the sv40 early promoter region (Bernoist, et al. Nature 290:304-10 (1981)); the promoter contained in the 3′ long terminal repeat (LTR) of Rous sarcoma virus (RSV) (Yamamoto, et al., 1980, cell 22:787-97); the herpes simplex virus thymidine kinase (HSV-TK) promoter (Wagner et al., Proc. Natl. Acad. Sci. USA, 78:1444-45 (1981)); the regulatory sequences of the metallothionine gene (Brinster et al. Nature 296:39-42 (1982)); prokaryotic expression vectors such as the beta-lactamase promoter (Villa-kamaroff et al., Proc. Natl. Acad. Sci. USA, 75:3727-31 (1978)); or, the tac promoter (Deboer et al. Proc. Natl. Acad. Sci. U.s.a., 80:21-25 (1983)). Tissue- and/or cell-type specific transcriptional control regions include, for example, the elastase I gene control region which is active in pancreatic acinar cells (Swift et al. Cell 38:639-46 (1984); Ornitz, et al. Cold Spring Harbor Symp. Quant. Biol. 50:399-409 (1986); Macdonald, et al. Hepatology 7:425-515 (1987)); the insulin gene control region which is active in pancreatic beta cells (Hanahan, et al. Nature 315:115-22 (1985)); the immunoglobulin gene control region which is active in lymphoid cells (Grosschedl et al. Cell 38:647-58 (1984); Adames et al. Nature 318:533-38 (1985); Alexander et al., Mol. Cell. Biol., 7:1436-44 (1987)); the mouse mammary tumor virus control region in testicular, breast, lymphoid and mast cells (Leder et al. Cell 45:485-95 (1986)); the albumin gene control region in liver (Pinkert et al. Genes and Devel. 1:268-76 (1987)); the alpha-feto-protein gene control region in liver (Krumlauf et al. Mol. Cell. Biol., 5:1639-48 (1985); Hammer et al. Science 235:53-58 (1987)); the alpha 1-antitrypsin gene control region in liver (Kelsey et al. Genes and Devel. 1:161-71 (1987)); the beta-globin gene control region in myeloid cells (Mogram et al. Nature 315:338-40 (1985); Kollias et al. Cell 46:89-94 (1986)); the myelin basic protein gene control region in oligodendrocyte cells in the brain (Readhead et al. Cell 48:703-12 (1987)); the myosin light chain-2 gene control region in skeletal muscle (Sani, et al. Nature 314:283-86 (1985)); the gonadotropic releasing hormone gene control region in the hypothalamus (Mason et al. Science 234:1372-78 (1986)), and the tyrosinase promoter in melanoma cells (Hart, et al. Semin. Oncol. February; 23(1): 154-8 (1996); Siders, et al. Cancer Gene Ther. September-October, 5(5): 281-91 (1998)), among others. Other suitable promoters are known in the art.

Nucleic acid delivery or transformation techniques that may be used in preparing and/or using the expression vectors of this disclosure can include DNA-ligand complexes, adenovirus-ligand-DNA complexes, direct injection of DNA, CaPO₄ precipitation, gene gun techniques, electroporation, and colloidal dispersion systems, among others. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome, which are artificial membrane vesicles useful as delivery vehicles in vitro and in vivo. RNA, DNA and intact virions can be encapsulated within the aqueous interior and be delivered to cells in a biologically active form (Fraley, R., et al. Trends Biochem. Sci., 6:77 (1981)). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

Strategies for improving the efficiency of nucleic acid-based immunization may also be used including, for example, the use of self-replicating viral replicons (Caley, et al. Vaccine, 17:3124-2135 (1999); Dubensky, et al. Mol. Med. 6:723-732 (2000); Leitner, et al. Cancer Res. 60:51-55 (2000)), codon optimization (Liu, et al. Mol. Ther., 1:497-500 (2000); Dubensky, supra; Huang, et al. J. Virol. 75:4947-4951 (2001)), in vivo electroporation (Widera, et al. J. Immunol. 164:4635-3640 (2000)), incorporation of CpG stimulatory motifs (Gurunathan, et al. Ann. Rev. Immunol. 18:927-974 (2000); Leitner, supra), sequences for targeting of the endocytic or ubiquitin-processing pathways (Thomson, et al. J. Virol. 72:2246-2252 (1998); Velders, et al. J. Immunol. 166:5366-5373 (2001)), prime-boost regimens (Gurunathan, supra; Sullivan, et al. Nature, 408:605-609 (2000); Hanke, et al. Vaccine, 16:439-445 (1998); Amara, et al. Science, 292:69-74 (2001)), and the use of mucosal delivery vectors such as Salmonella (Darji, et al. Cell, 91:765-775 (1997); Woo, et al. Vaccine, 19:2945-2954 (2001)). Other methods are known in the art, some of which are described below.

In other embodiments, it may be advantageous to combine or include within the compositions or recombinant vectors additional polypeptides, peptides or polynucleotides encoding one or more polypeptides or peptides that function as “co-stimulatory” component(s). Such co-stimulatory components may include, for example, cell surface proteins, cytokines or chemokines in a composition of the present invention. The co-stimulatory component may be included in the composition as a polypeptide or peptide, or as a polynucleotide encoding the polypeptide or peptide, for example. Suitable co-stimulatory molecules may include, for example, polypeptides that bind members of the CD28 family (i.e., CD28, ICOS; Hutloff, et al. Nature 1999, 397:263-265; Peach, et al. J Exp Med 1994, 180:2049-2058) such as the CD28 binding polypeptides B7.1 (CD80; Schwartz, 1992; Chen et al, 1992; Ellis, et al. J. Immunol., 156(8): 2700-9) and B7.2 (CD86; Ellis, et al. J. Immunol., 156(8): 2700-9); polypeptides which bind members of the integrin family (i.e., LFA-1 (CD11a/CD18); Sedwick, et al. J Immunol 1999, 162:1367-1375; Wülfing, et al. Science 1998, 282:2266-2269; Lub, et al. Immunol Today 1995, 16:479-483) including members of the ICAM family (i.e., ICAM-1, -2 or -3); polypeptides which bind CD2 family members (i.e., CD2, signalling lymphocyte activation molecule (CDw150 or “SLAM”; Aversa, et al. J Immunol 1997, 158:4036-4044) such as CD58 (LFA-3; CD2 ligand; Davis, et al. Immunol Today 1996, 17:177-187) or SLAM ligands (Sayos, et al. Nature 1998, 395:462-469); polypeptides which bind heat stable antigen (HSA or CD24; Zhou, et al. Eur J Immunol 1997, 27:2524-2528); polypeptides which bind to members of the TNF receptor (TNFR) family (i.e., 4-1BB (CD137; Vinay, et al. Semin Immunol 1998, 10:481-489)), OX40 (CD134; Weinberg, et al. Semin Immunol 1998, 10:471-480; Higgins, et al. J Immunol 1999, 162:486-493), and CD27 (Lens, et al. Semin Immunol 1998, 10:491-499)) such as 4-1BBL (4-1BB ligand; Vinay, et al. Semin Immunol 1998, 10:481-48; DeBenedette, et al. J Immunol 1997, 158:551-559), TNFR associated factor-1 (TRAF-1; 4-1BB ligand; Saoulli, et al. J Exp Med 1998, 187:1849-1862, Arch, et al. Mol Cell Biol 1998, 18:558-565), TRAF-2 (4-1BB and OX40 ligand; Saoulli, et al. J Exp Med 1998, 187:1849-1862; Oshima, et al. Int Immunol 1998, 10:517-526, Kawamata, et al. J Biol Chem 1998, 273:5808-5814), TRAF-3 (4-1BB and OX40 ligand; Arch, et al. Mol Cell Biol 1998, 18:558-565; Jang, et al. Biochem Biophys Res Commun 1998, 242:613-620; Kawamata S, et al. J Biol Chem 1998, 273:5808-5814), OX40L (OX40 ligand; Gramaglia, et al. J Immunol 1998, 161:6510-6517), TRAF-5 (OX40 ligand; Arch, et al. Mol Cell Biol 1998, 18:558-565; Kawamata, et al. J Biol Chem 1998, 273:5808-5814), and CD70 (CD27 ligand; Couderc, et al. Cancer Gene Ther., 5(3): 163-75). CD154 (CD40 ligand or “CD40L”; Gurunathan, et al. J. Immunol., 1998, 161:4563-4571; Sine, et al. Hum. Gene Ther., 2001, 12:1091-1102) Other co-stimulatory molecules may also be suitable for use as disclosure herein.

One or more cytokines may also be suitable co-stimulatory components or “adjuvants”, either as polypeptides or being encoded by polynucleotides contained within the compositions of the present invention (Parmiani, et al. Immunol Lett 2000 Sep. 15; 74(1): 41-4; Berzofsky, et al. Nature Immunol. 1:209-219). Suitable cytokines include, for example, interleukin-2 (IL-2) (Rosenberg, et al. Nature Med. 4:321-327 (1998)), IL-4, IL-7, IL-12 (reviewed by Pardoll, 1992; Harries, et al. J. Gene Med. 2000 July-August; 2(4): 243-9; Rao, et al. J. Immunol. 156:3357-3365 (1996)), IL-15 (Xin, et al. Vaccine, 17:858-866, 1999), IL-16 (Cruikshank, et al. J. Leuk Biol. 67(6): 757-66, 2000), IL-18 (J. Cancer Res. Clin. Oncol. 2001. 127(12): 718-726), GM-CSF (CSF (Disis, et al. Blood, 88:202-210 (1996)), tumor necrosis factor-alpha (TNF-α), or interferon-gamma (INF-γ). Chemokines may also be utilized. For example, fusion proteins comprising CXCL10 (IP-10) and CCL7 (MCP-3) fused to a tumor self-antigen have been shown to induce anti-tumor immunity (Biragyn, et al. Nature Biotech. 1999, 17:253-258). The chemokines CCL3 (MIP-1α) and CCL5 (RANTES) (Boyer, et al. Vaccine, 1999, 17 (Supp. 2): S53-S64) may also be of use. Other suitable chemokines are known in the art. It is also known in the art that suppressive or negative regulatory immune mechanisms may be blocked, resulting in enhanced immune responses. For instance, treatment with anti-CTLA-4 (Shrikant, et al. Immunity, 1996, 14:145-155; Sutmuller, et al. J. Exp. Med., 2001, 194:823-832), anti-CD25 (Sutmuller, supra), anti-CD4 (Matsui, et al. J. Immunol., 1999, 163:184-193), the fusion protein IL13Rα2-Fc (Terabe, et al. Nature Immunol., 2000, 1:515-520), and combinations thereof (i.e., anti-CTLA-4 and anti-CD25, Sutmuller, supra) have been shown to upregulate anti-tumor immune responses and would be suitable. Other cytokines, chemokines, suppressive and/or regulatory agents may also be suitable for use as disclosed herein.

An immunogen (preferably administered as a modified NYVAC vector encoding one or more coronavirus spike protein and/or derivatives thereof) may also be administered in combination with one or more adjuvants to boost the immune response. Adjuvants may also be included to stimulate or enhance the immune response. Non-limiting examples of suitable adjuvants include those of the gel-type (i.e., aluminum hydroxide/phosphate (“alum adjuvants”), calcium phosphate), of microbial origin (muramyl dipeptide (MDP)), bacterial exotoxins (cholera toxin (CT), native cholera toxin subunit B (CTB), E. coli labile toxin (LT), pertussis toxin (PT), CpG oligonucleotides, BCG sequences, tetanus toxoid, monophosphoryl lipid A (MPL) of, for example, E. coli, Salmonella minnesota, Salmonella typhimurium, or Shigella exseri), particulate adjuvants (biodegradable, polymer microspheres), immunostimulatory complexes (ISCOMs)), oil-emulsion and surfactant-based adjuvants (Freund's incomplete adjuvant (FIA), microfluidized emulsions (MF59, SAF), saponins (QS-21)), synthetic (muramyl peptide derivatives (murabutide, threony-MDP), nonionic block copolymers (L121), polyphosphazene (PCCP), synthetic polynucleotides (poly A: U, poly I:C), thalidomide derivatives (CC-4407/ACTIMID)), RH3-ligand, or polylactide glycolide (PLGA) microspheres, among others. Fragments, homologs, derivatives, and fusions to any of these toxins are also suitable, provided that they retain adjuvant activity. Suitable mutants or variants of adjuvants are described, e.g., in WO 95/17211 (Arg-7-Lys CT mutant), WO 96/6627 (Arg-192-Gly LT mutant), and WO 95/34323 (Arg-9-Lys and Glu-129-Gly PT mutant). Additional LT mutants that can be used in the methods and compositions of the invention include, e. g., Ser-63-Lys, Ala-69-Gly, Glu-110-Asp, and Glu-112-Asp mutants. As an example, metallic salt adjuvants such alum adjuvants are well-known in the art as providing a safe excipient with adjuvant activity. The mechanism of action of these adjuvants are thought to include the formation of an antigen depot such that antigen may stay at the site of injection for up to 3 weeks after administration, and also the formation of antigen/metallic salt complexes which are more easily taken up by antigen presenting cells. In addition to aluminium, other metallic salts have been used to adsorb antigens, including salts of zinc, calcium, cerium, chromium, iron, and beryllium. The hydroxide and phosphate salts of aluminium are the most common. Formulations or compositions containing aluminium salts, antigen, and an additional immunostimulant are known in the art. An example of an immunostimulant is 3-de-O-acylated monophosphoryl lipid A (3D-MPL). Other suitable adjuvants are also well-known in the art.

Any of these components may be used alone or in combination with other agents. For instance, it has been shown that a combination of CD80, ICAM-1 and LFA-3 (“TRICOM”) may potentiate anti-cancer immune responses (Hodge, et al. Cancer Res. 59:5800-5807 (1999). Other effective combinations include, for example, IL-12+GM-CSF (Ahlers, et al. J. Immunol., 158:3947-3958 (1997); Iwasaki, et al. J. Immunol. 158:4591-4601 (1997)), IL-12+GM-CSF+TNF-α (Ahlers, et al. Int. Immunol. 13:897-908 (2001)), CD80+IL-12 (Fruend, et al. Int. J. Cancer, 85:508-517 (2000); Rao, et al. supra), and CD86+GM-CSF+IL-12 (Iwasaki, supra). One of skill in the art would be aware of additional combinations useful in carrying out the present invention. In addition, the skilled artisan would be aware of additional reagents or methods that may be used to modulate such mechanisms. These reagents and methods, as well as others known by those of skill in the art, may be utilized as disclosed herein.

Other agents that may be utilized in conjunction with the compositions and methods provided herein include anti-coronavirus and/or anti-influenza agents (e.g., antibiotics, anti-viral, or anti-parasitic (e.g., ivermectin) medications, anti-IL6). In some embodiments, the additional agent can reduce inflammation or other symptoms associated with coronavirus infection and/or can function to reduce the viability of the coronavirus in the host. For instance, suitable that can be included as a pre-treatment or co-treatment (e.g., administered essentially with or shortly after the recombinant expression vectors disclosed herein) can include: anti-IL-1α agents such as anakinra (Kineret®), canakinumab (Ilaris®), rilonacept (Arcalyst®); anti-IL5 agents such as mepolizumab (GlaxoSmithKline) or benralizumab (Fasenra); anti-IL6 agents such as tocilzumab (Actemra), sarilumab (Kevzara), siltuximab (Sylvant); anti-IL12 agents such as briakinumab (ABT-874, Abbott) or ustekinumab; anti-IL17 agents such as brodalumab (Siliq; Amgen), ixekizumab (Taltz®, Eli Lilly), or secukinumab (Cosentyx; Novartis); anti-TNF-alpha agents such as inflixibmab (Remicade), adalimumab (Humira), certolizumab pegol (Cimzia), golimumab (Simponi), etanercept (Enbrel), thalidomide (Immunoprin), lenalidomide (Revlimid), pomalidomide (Pomalyst, Imnovid), a xanthine derivative (e.g., pentoxifylline), or bupropion; anti-GM-CSF agent such as otilimab (MOR103, GSK3196165); and/or, one or more anti-SARS drugs such as hydroxychloroquine, azythromycin, remdesivir, and/or ivermectin. Preferred, exemplary anti-influenza agents can include oseltamivir phosphate (available as a generic version or under the trade name Tamiflu®), zanamivir (trade name Relenza®), peramivir (trade name Rapivab®), and, baloxavir marboxil (trade name Xofluza®). Other agents may also be suitable as may be determined by those of ordinary skill in the art.

Administration of a composition of the present invention to a host may be accomplished using any of a variety of techniques known to those of skill in the art. The composition(s) may be processed in accordance with conventional methods of pharmacy to produce medicinal agents for administration to patients, including humans and other mammals (i.e., a “pharmaceutical composition”). The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of a nucleic acid or polypeptide used to observe the desired therapeutic effect (e.g., induce or enhance and immune response). Injectable preparations, such as sterile injectable aqueous or oleaginous suspensions, may be formulated according to known methods using suitable dispersing or wetting agents and suspending agents. The injectable preparation may also be a sterile injectable solution or suspension in a non-toxic parenterally acceptable diluent or solvent. Suitable vehicles and solvents that may be employed are water, Ringer's solution, and isotonic sodium chloride solution, among others. For instance, a viral vector such as a poxvirus may be prepared in 0.4% NaCl or a Tris-HCl buffer, with or without a suitable stabilizer such as lactoglutamate, and with or without freeze drying medium. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables. Pharmaceutical compositions may take any of several forms and may be administered by any of several routes. The compositions are administered via a parenteral route (e.g., intradermal, intramuscular, subcutaneous, skin scarification) to induce an immune response in the host. Alternatively, the composition may be administered directly into a lymph node (intranodal) or tumor mass (i.e., intratumoral administration). Preferred embodiments of administrable compositions include, for example, nucleic acids, viral particles, or polypeptides in liquid preparations such as suspensions, syrups, or elixirs. Preferred injectable preparations include, for example, nucleic acids or polypeptides suitable for parental, subcutaneous, intradermal, intramuscular or intravenous administration such as sterile suspensions or emulsions. For example, a naked DNA molecule and/or recombinant poxvirus may separately or together be in admixture with a suitable carrier, diluent, or excipient such as sterile water, physiological saline, glucose or the like. The composition may also be provided in lyophilized form for reconstituting, for instance, in isotonic aqueous, saline buffer. In addition, the compositions can be co-administered or sequentially administered with one another, with one or more other antiviral compounds, and/or compounds that reduce or alleviate ill effects of coronavirus infection or the effects of such agents.

The pharmaceutical composition is preferably made in the form of a dosage unit containing a given amount of DNA, viral vector particles (preferably modified NYVAC vector encoding at least one coronavirus antigen or derivative thereof), and/or expressed polypeptide, for example. A suitable daily dose for a human or other mammal may vary widely depending on the condition of the patient and other factors, but, once again, can be determined using routine methods. The compositions are administered to a patient in a form and amount sufficient to elicit a therapeutic effect. Amounts effective for this use will depend on various factors, including, for example, the particular composition of the vaccine regimen administered, the manner of administration, the stage and severity of the disease, the general state of health of the patient, and the judgment of the prescribing physician. The dosage regimen for immunizing a host or otherwise treating a disorder or a disease with a composition of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. In general, recombinant viruses may be administered in compositions in an amount of about 10⁴ to about 10⁹ pfu per inoculation; often about 10⁴ pfu to about 10⁶ pfu, or as shown in the Examples, 10⁷ to 10³ pfu. Higher dosages such as about 10⁴ pfu to about 10¹⁰ pfu, e.g., about 10⁵ pfu to about 10⁹ pfu, or about 10⁶ pfu to about 10⁸ pfu, or about 10⁷ pfu can also be employed. Another measure commonly used is DICC₅₀; suitable DICC₅₀ ranges for administration include about 10¹, about 10², about 10³, about 10⁴, about 10⁵, about 10⁶, about 10⁷, about 10⁸, about 10⁹, about 10¹⁰ DICC₅₀. Ordinarily, suitable quantities of plasmid or naked DNA are about 1 μg to about 100 mg, about 1 mg, about 2 mg, but lower levels such as 0.1 to 1 mg or 1-10 μg may be employed. Actual dosages of such compositions can be readily determined by one of ordinary skill in the field of vaccine technology. For the purposes of this disclosure, a suitable administration scheme (e.g., dosage amount and schedule) is one that induces an anti-coronavirus immune response in a host animal (e.g., as shown in the examples).

As previously mentioned, while the compositions described herein may be administered as the sole active pharmaceutical agent, they can also be used in combination with one or more other compositions or agents (i.e., other immunogens, co-stimulatory molecules, adjuvants). When administered as a combination, the individual components can be formulated as separate compositions administered at the same time or different times, or the components can be combined as a single composition. In one embodiment, a method of administering to a host a first form of an immunogen and subsequently administering a second form of the immunogen, wherein the first and second forms are different, and wherein administration of the first form prior to administration of the second form enhances the immune response resulting from administration of the second form relative to administration of the second form alone, is provided. Also provided are compositions for administration to the host. For example, a two-part immunological composition where the first part of the composition comprises a first form of an immunogen and the second part comprises a second form of the immunogen, wherein the first and second parts are administered separately from one another such that administration of the first form enhances the immune response against the second form relative to administration of the second form alone, is provided. The immunogens, which may be the same or different, are preferably derived from the infectious agent or other source of immunogens. The multiple immunogens may be administered together or separately, as a single or multiple compositions, or in single or multiple recombinant vectors.

A kit comprising a composition of the present invention is also provided. The kit can include a separate container containing a suitable carrier, diluent or excipient. The kit may also include additional components for simultaneous or sequential-administration. In one embodiment, such a kit may include a first form of an immunogen and a second form of the immunogen. Additionally, the kit can include instructions for mixing or combining ingredients and/or administration. A kit may provide reagents for performing screening assays, such as one or more PCR primers, hybridization probes, and/or biochips, for example.

This disclosure discloses several embodiments. In preferred embodiments, this disclosure provides the following aspects:
1. A recombinant NYVAC vector comprising within its genome a polynucleotide encoding:
   a) C7L (SEQ ID NO: 17) and K1L (SEQ ID NO: 27), wherein the C7L (SEQ ID NO: 17) and K1L (SEQ ID NO: 27) coding sequences are adjacent to one another in the genome: or,
   b) C7L (SEQ ID NO. 17) and K1L (SEQ ID NO. 27), and optionally at least one of C1L (SEQ ID NO. 5), C2L (SEQ ID NO. 7), C3L (SEQ ID NO. 9), C4L (SEQ ID NO. 11), C5L (SEQ ID NO. 13), C6L (SEQ ID NO. 15), NIL (SEQ ID NO. 19). N2L (SEQ ID NO. 21), M1L (SEQ ID NO. 23), and M2L (SEQ ID NO, 25); wherein the C7L (SEQ ID NO. 17) and the and K1L (SEQ ID NO. 27) are positioned adjacent to C8L and K2L, respectively, in the genome; or,
   c) a polypeptide having at least approximately 90% identity to C7L (SEQ ID NO. 17), a polypeptide having at least approximately 90% identity to K1L (SEQ ID NO. 27), and at least one polypeptide having at least approximately 90% identity to a polypeptide selected from the group consisting of C1L (SEQ ID NO. 5), C2L (SEQ ID NO. 7), C3L (SEQ ID NO. 9), C4L (SEQ ID NO. 11), C5L (SEQ ID NO. 13), C6L (SEQ ID NO. 15), NIL (SEQ ID NO. 19), N2L (SEQ ID NO. 21), M1L (SEQ ID NO. 23), and M2L (SEQ ID NO. 25), wherein the at least one polynucleotide encoding the polypeptide having at least approximately 90% identity to C7L (SEQ ID NO. 17) is positioned adjacent to the C8L coding sequence in the genome and the at least one polynucleotide encoding the polypeptide having at least approximately 90% identity to K1L (SEQ ID NO. 27) is positioned adjacent to the K2L coding sequence in the genome; or,
   d) a first polypeptide having at least about 90% identity to C7L (SEQ ID NO: 17), and a second polypeptide having at least about 90% identity to K1L (SEQ ID NO: 27), wherein polynucleotides encoding the first polypeptide and second polypeptide are positioned adjacent to one another in the genome;
   wherein the recombinant NYVAC vector further comprises:
      one or more modifications of at least one polynucleotide encoding B8R (SEQ ID NO. 1) and B19R (SEQ ID NO. 3) that renders the vector unable to express the B8R and B19R polypeptides; and,
      a polynucleotide encoding at least one coronavirus and/or influenza immunogen.
2. The recombinant NYVAC vector of aspect 1 wherein the at least one coronavirus is selected from the group consisting of SARS-CoV-2 or a variant thereof (e.g., alpha, beta, gamma, delta, omicron, epsilon, zeta, eta, theta, iota, kappa, lambda, mu, cluster 5, or lineage B.1.617).
3. The recombinant NYVAC vector of aspect 1 or 2 wherein the at least one coronavirus antigen is the psfSpike polypeptide (SEQ ID NO: 31).
4. The recombinant NYVAC vector of aspect 2 or 3 wherein the psfSpike polypeptide is encoded by SEQ ID NO: 32.
5. The recombinant NYVAC vector of aspect 1 encoding at least one immunogen of an influenza virus of the Influenza A, optionally any one or more of Influenza A subtypes H1 through H18 and N1 through N11 (e.g., subtypes A (H1N1 (e.g., 2009 H1N1), H3N2); Influenza B, optionally any one or more of Influenza B subtype VIA, V1A.1, V1A.2, or V1A.3; or the Yamagata lineage, optionally any one or more of the Y1, Y2, or Y3 subtypes.
6. The recombinant NYVAC vector of aspect 1 and/or 2 wherein the polynucleotide encodes at least one influenza hemagglutinin (e.g., (A/Thailand/1 (KAN-1)/2004, GenBank AY555150)), neuraminidase, nucleoprotein ((NP (A/Thailand/1 (KAN-1)/2004, GenBank AAV35112 and A/PR/8/34, GenBank AAM75159), and/or extracellular domain of matrix 2 ion channel protein ((A/Thailand/1 (KAN-1)/2004, GenBank AAV35111)).
7. The recombinant NYVAC vector of any one of aspects 1-6 further comprising a polynucleotide encoding ATV eIF2αH (SEQ ID NO. 29).
8. The recombinant NYVAC vector of any one of aspects 1-7, the vector further comprising a polynucleotide encoding at least one additional immunogen.
9. A composition comprising a recombinant NYVAC vector of any prior aspect and a pharmaceutically acceptable carrier.
10. A method of immunizing a host against infection by coronavirus and/or preventing and/or reducing the symptoms of infection by coronavirus, the method comprising administering to the host a composition of aspect 9 to the host.
11. The method of aspect 10 wherein administration of the composition affects cells of the host immune system as determined by detecting a change in at least one immune cells characteristic selected from the group consisting of maturation, proliferation, improved direct presentation of antigen, improved cross-presentation of antigen, and an activated immunomodulatory gene expression profile.
12. The method of aspect 11 wherein the immune cells comprise one or more cell types selected from the group consisting of dendritic cells, lymphocytes, monocytes, macrophages, natural killer cells, and granulocytes.
13. The method of aspect 12 wherein the lymphocytes are cytotoxic T cells.
14. The method of aspect 12 wherein the lymphocytes are B cells.
15. The method of aspect 12 wherein the lymphocytes are cytotoxic T cells and B cells.
16. The method of any one of aspects 10-15 wherein the method induces a protective immune response.
17. A method of manufacturing a recombinant NYVAC vector encoding a coronavirus antigen, the method comprising, in any order:
   a) deleting the region of the NYVAC genome encoding C1L (SEQ ID NO: 5), C2L (SEQ ID NO: 7), C3L (SEQ ID NO: 9), C4L (SEQ ID NO: 11), C5L (SEQ ID NO: 13), C6L (SEQ ID NO: 15), C7L (SEQ ID NO: 17), NIL (SEQ ID NO: 19), N2L (SEQ ID NO: 21), M1L (SEQ ID NO: 23), M2L (SEQ ID NO: 25), and K1L (SEQ ID NO: 27) therefrom;
   b) deleting the regions of the NYVAC genome encoding B8R (SEQ ID NO. 1) and B19R (SEQ ID NO. 3); and,
   c) inserting into the NYVAC genome at least one polynucleotide encoding at least one coronavirus or influenza immunogen.
18. The method of aspect 17 wherein the at least one coronavirus is selected from the group consisting of SARS-CoV-2 or a variant thereof (e.g., alpha, beta, gamma, delta, omicron, epsilon, zeta, eta, theta, iota, kappa, lambda, mu, cluster 5, or lineage B.1.617).
19. The method of aspect 18 wherein the at least one coronavirus immunogen is the psfSpike polypeptide (SEQ ID NO: 31).
20. The method of aspect 19 wherein the psfSpike polypeptide is encoded by SEQ ID NO: 32.
21. The method of aspect 17 encoding at least one immunogen of an influenza virus of the Influenza A, optionally any one or more of Influenza A subtypes H1 through H18 and N1 through N11 (e.g., subtypes A (H1N1 (e.g., 2009 H1N1), H3N2); Influenza B, optionally any one or more of Influenza B subtype VIA, V1A.1, V1A.2, or V1A.3; or the Yamagata lineage, optionally any one or more of the Y1, Y2, or Y3 subtypes.
22. The method of aspect 121 wherein the polynucleotide encodes at least one influenza hemagglutinin (e.g., (A/Thailand/1 (KAN-1)/2004, GenBank AY555150)), neuraminidase, nucleoprotein ((NP (A/Thailand/1 (KAN-1)/2004, GenBank AAV35112 and A/PR/8/34, GenBank AAM75159), and/or extracellular domain of matrix 2 ion channel protein ((A/Thailand/1 (KAN-1)/2004, GenBank AAV35111)).

Other aspects are also provided by this disclosure as would be understood by those of ordinary skill in the art.

All references cited within this application are incorporated by reference. A better understanding of the present invention and of its many advantages will be had from the following examples, given by way of illustration.

### EXAMPLES

### Example 1

### Modified Expression Vectors Encoding Coronavirus Antigens

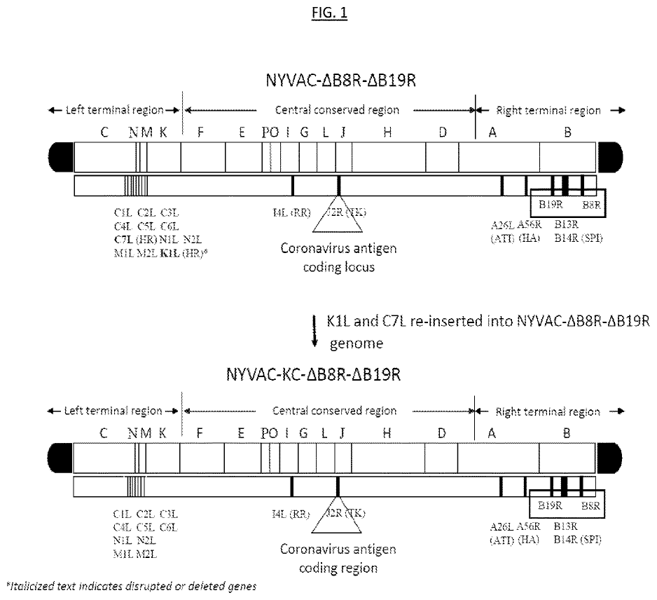

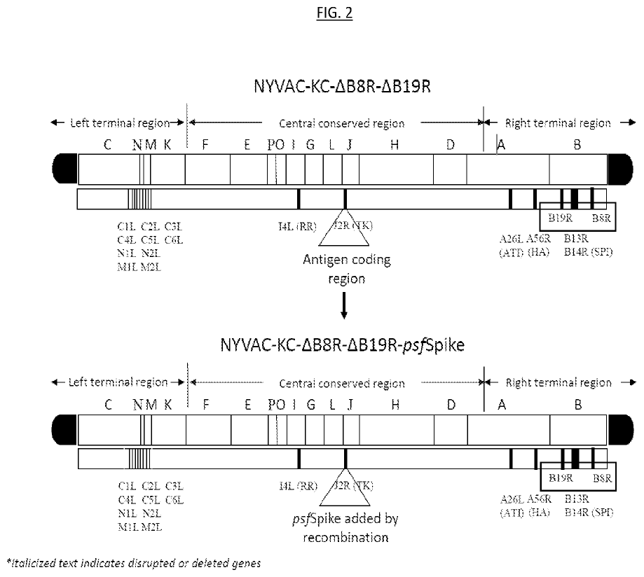

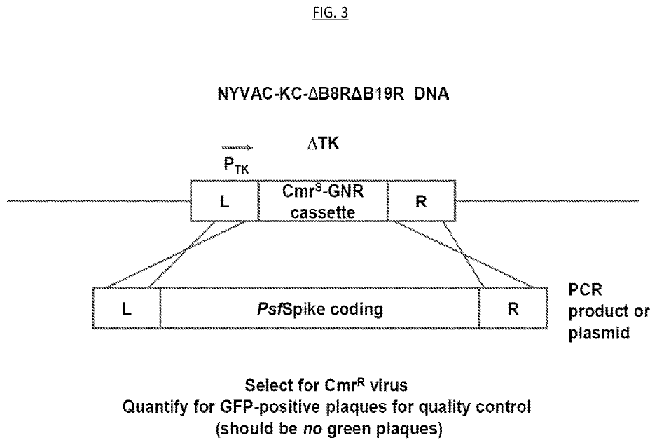

The construction and characteristics of the modified expression vector NYVAC-KC-ΔB8R-ΔB19R utilized herein are described in U.S. Pat. No. 9,670,506 filed on Apr. 10, 2010 (Pantaleo, et al.) As shown in U.S. Pat. No. 9,670,506, deletion of the B8R (SEQ ID Nos. 1-2) and B19 (SEQ ID Nos. 13-14) from a first modified NYVAC vector was shown to provide improved immunogenicity characteristics to the vector. To create the first modified NYVAC, the parental NYVAC vector was modified by deleting a non-essential region of the vaccinia virus genome containing C1L (SEQ ID Nos. 5-6), C2L (SEQ ID Nos. 7-8), C3L (SEQ ID Nos. 9-10), C4L (SEQ ID Nos. 11-12), C5L (SEQ ID Nos. 13-14), C6L (SEQ ID Nos. 15-16), C7L (SEQ ID Nos. 17-18), NIL (SEQ ID Nos. 19-20), N2L (SEQ ID Nos. 21-22), M1L (SEQ ID Nos. 23-24), M2L (SEQ ID Nos. 25-26), and K1L (SEQ ID Nos. 27-28) (see Tartaglia, et al. Virology, 188:217-232 (1992)). Deletion of genes in this region rendered NYVAC replication-defective in human cells. A second modified NYVAC vector was produced that includes C7L and K1L inserted back into the region of the NYVAC genome from which C1L, C2L, C3L, C4L, C5L, C6L, NIL, N2L, M1L, and M2L were deleted. As shown in U.S. Pat. No. 9,670,506, replication competence of NYVAC was restored by re-insertion of these two host range genes C7L and K1L into NYVAC to produce NYVAC-KC. B8R and B19R were deleted from NYVAC-KC to produce the modified vector NYVAC-KC-ΔB8R-ΔB19R (also referred to as NYVAC-KC-AA). Thus, the vector used in these examples is a modified NYVAC lacking the B8R and B19R open reading frames and including the C7L and K1L open reading frames in the genome. As shown in U.S. Pat. No. 9,670,506, the NYVAC-KC-ΔB8R-ΔB19R vector was used to induce a potent immune response against HIV antigens by incorporating HIV antigens therein. This disclosure relates to the use of the modified NYVAC vector NYVAC-KC-ΔB8R-ΔB19R vector to induce an immune response against the coronavirus spike protein. The coronavirus spike protein used in this example is that of COVID-19 and has the amino acid sequence of SEQ ID NO: 31 (encoded by SEQ ID NO: 32). The construction of NYVAC-KC-ΔB8R-ΔB19R vector, including the modifications described in U.S. Pat. No. 9,670,506 and the insertion of the coronavirus spike protein coding region (psfSpike) is summarized in FIGS. 1 and 2. A selection cassette, PGNR, was inserted into the TK locus of NYVAC-KC-ΔB8R-ΔB19R. The cassette encodes an E. coli gyrase/PKR fusion protein that confers coumermycin (cmr) sensitivity, and a neo^{R} gene fused to the green fluorescent protein (GFP) gene. The cassette is flanked by the TK locus arms. Plaques formed by this virus are green, and sensitive to cmr. The psfSpike gene, flanked by TK locus arms, replaces the cassette through in vivo recombination of the TK locus arms when cells are infected with NYVAC-KC-ΔB8R-ΔB19R and transfected with the plasmid carrying the TK locus arms and psfSpike gene (FIG. 3) to produce the NYVAC-KC-ΔB8R-ΔB19R-psfSpike vector (abbreviated “NYVAC-KC-ΔΔ-psfSpike”). A vector including the B8R and B19R open reading frames as well as the K1L and C7L genes into the NYVAC genome (“NYVAC-KC-psfSpike”).

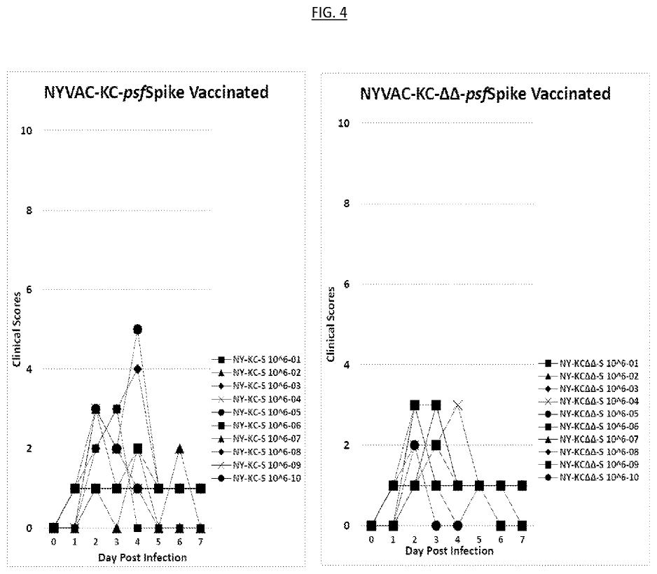

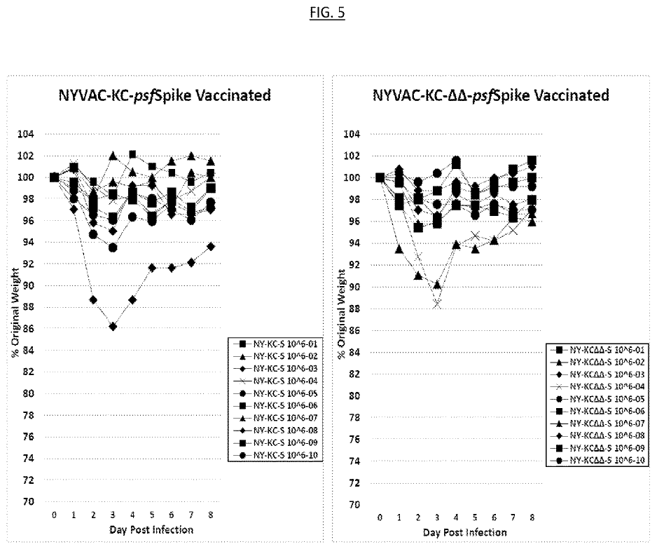

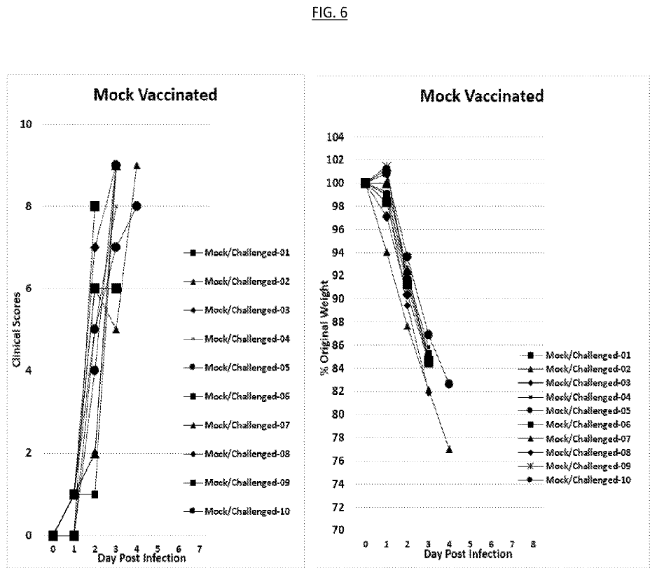

To compare the effectiveness of the NYVAC-KC-psfSpike and the NYVAC-KC-AA-psfSpike vectors, eight-week-old Balb/C mice were either mock vaccinated (test mice), or vaccinated with 10⁶ pfu NYVAC-KC-psfSpike or 10⁶ pfu NYVAC-KC-ΔΔ-psfSpike. All vaccinations were by scarification. All animals received a boost vaccination at 28 days post-prime vaccination; challenge was 14 days post-boost vaccination. Mice were challenged with 2×10³ pfu of mouse-adapted SARS2-N501Y_{MA30}. Clinical scores are based on evaluation of visible symptoms (e.g., hunching, lethargy, and normal/abnormal breathing), plus a score for weight loss. A higher clinical score is less desirable as it indicates symptoms of illness. FIG. 4 shows to which the NYVAC-KC-psfSpike vector was administered exhibited higher (i.e., less desirable) clinical scores following challenge with mouse-adapted SARS2-N501Y_{MA30} than mice to which the NYVAC-KC-ΔΔ-psfSpike vector was administered (“Days Post Infection”). FIG. 5 shows the effects of the administration of the NYVAC-KC-psfSpike and the NYVAC-KC-AA-psfSpike vectors on the weight of tested animals. FIG. 6 illustrates the much higher clinical scores and decreased weight of challenged animals that were not vaccinated with either of the NYVAC-KC-psfSpike or NYVAC-KC-ΔΔ-psfSpike vectors.

All documents cited in this disclosure are hereby incorporated into this disclosure in their entirety. While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed:

1. A recombinant NYVAC vector comprising within its genome a polynucleotide encoding:
a) C7L (SEQ ID NO: 17) and K1L (SEQ ID NO: 27), wherein the C7L (SEQ ID NO: 17) and K1L (SEQ ID NO; 27) coding sequences are adjacent to one another in the genome; or,
b) C7L (SEQ ID NO. 17) and K1L (SEQ ID NO. 27), and optionally at least one of C1L (SEQ ID NO. 5), C2L (SEQ ID NO. 7), C3L (SEQ ID NO. 9), C4L (SEQ ID NO. 11), C5L (SEQ ID NO. 13), C6L (SEQ ID NO. 15), NIL (SEQ ID NO. 19), N2L (SEQ ID NO. 21), M1L (SEQ ID NO. 23), and M2L (SEQ ID NO. 25): wherein the C7L (SEQ ID NO. 17) and the and K1L (SEQ ID NO. 27) are positioned adjacent to C8L and K2L, respectively, in the genome; or,
c) a polypeptide having at least approximately 90% identity to C7L (SEQ ID NO. 17), a polypeptide having at least approximately 90% identity to K1L (SEQ ID NO. 27), and at least one polypeptide having at least approximately 90% identity to a polypeptide selected from the group consisting of C1L (SEQ ID NO. 5), C2L (SEQ ID NO. 7), C3L (SEQ ID NO. 9), C4L (SEQ ID NO. 11), C5L (SEQ ID NO. 13), C6L (SEQ ID NO. 15), NIL (SEQ ID NO. 19), N2L (SEQ ID NO. 21), M1L (SEQ ID NO. 23), and M2L (SEQ ID NO. 25), wherein the at least one polynucleotide encoding the polypeptide having at least approximately 90% identity to C7L (SEQ ID NO. 17) is positioned adjacent to the C8L coding sequence in the genome and the at least one polynucleotide encoding the polypeptide having at least approximately 90% identity to K1L (SEQ ID NO. 27) is positioned adjacent to the K2L coding sequence in the genome; or,
d) a first polypeptide having at least about 90% identity to C7L (SEQ ID NO: 17), and a second polypeptide having at least about 90% identity to K1L (SEQ ID NO: 27), wherein polynucleotides encoding the first polypeptide and second polypeptide are positioned adjacent to one another in the genome;
wherein the recombinant NYVAC vector further comprises:
one or more modifications of at least one polynucleotide encoding B8R (SEQ ID NO. 1) and B19R (SEQ ID NO. 3) that renders the vector unable to express the B8R and B19R polypeptides; and,
a polynucleotide encoding at least one coronavirus and/or influenza immunogen.

2. The recombinant NYVAC vector of aspect 1 wherein the at least one coronavirus is selected from the group consisting of SARS-CoV-2, optionally a variant thereof selected from the group consisting of alpha, beta, gamma, delta, omicron, epsilon, zeta, eta, theta, iota, kappa, lambda, mu, cluster 5, and lineage B.1.617.

3. The recombinant NYVAC vector of claim 1 or 2 wherein the at least one coronavirus antigen is the psfSpike polypeptide (SEQ ID NO: 31).

4. The recombinant NYVAC vector of claim 3 wherein the psfSpike polypeptide is encoded by SEQ ID NO: 32.

5. The recombinant NYVAC vector of claim 1 encoding at least one immunogen of an influenza virus of the Influenza A, optionally any one or more of Influenza A subtypes H1 through H18 and N1 through N11 (e.g., subtypes A (H1N1 (e.g., 2009 H1N1), H3N2); Influenza B, optionally any one or more of Influenza B subtype VIA, V1A.1, V1A.2, or V1A.3; or the Yamagata lineage, optionally any one or more of the Y1, Y2, or Y3 subtypes.

6. The recombinant NYVAC vector of claim 1 or 5 wherein the polynucleotide encodes at least one influenza hemagglutinin, GenBank AY555150, neuraminidase, nucleoprotein, GenBank AAV35112, GenBank AAM75159, and/or extracellular domain of matrix 2 ion channel protein, and, GenBank AAV35111.

7. The recombinant NYVAC vector of claim 1 further comprising a polynucleotide encoding ATV eIF2αH (SEQ ID NO. 29).

8. The recombinant NYVAC vector of claim 1, the vector further comprising a polynucleotide encoding at least one additional immunogen.

9. A composition comprising a recombinant NYVAC vector of claim 1 and a pharmaceutically acceptable carrier.

10. A method of immunizing a host against infection by coronavirus and/or influenza and/or preventing and/or reducing the symptoms of infection by coronavirus and/or influenza, respectively, the method comprising administering to the host a composition of claim 9 to the host.

11. The method of claim 10 wherein administration of the composition affects cells of the host immune system as determined by detecting a change in at least one immune cells characteristic selected from the group consisting of maturation, proliferation, improved direct presentation of antigen, improved cross-presentation of antigen, and an activated immunomodulatory gene expression profile.

12. The method of claim 11 wherein the immune cells comprise one or more cell types selected from the group consisting of dendritic cells, lymphocytes, monocytes, macrophages, natural killer cells, and granulocytes.

13. The method of claim 12 wherein the lymphocytes are cytotoxic T cells.

14. The method of claim 13 wherein the lymphocytes are B cells.

15. The method of claim 14 wherein the lymphocytes are cytotoxic T cells and B cells.

16. The method of any one of claims 10-15 wherein the method induces a protective immune response.

17. A method of manufacturing a recombinant NYVAC vector encoding a coronavirus antigen, the method comprising, in any order:
d) deleting the region of the NYVAC genome encoding C1L (SEQ ID NO: 5), C2L (SEQ ID NO: 7), C3L (SEQ ID NO: 9), C4L (SEQ ID NO: 11), C5L (SEQ ID NO: 13), C6L (SEQ ID NO: 15), C7L (SEQ ID NO: 17), NIL (SEQ ID NO: 19), N2L (SEQ ID NO: 21), M1L (SEQ ID NO: 23), M2L (SEQ ID NO: 25), and K1L (SEQ ID NO: 27) therefrom;
e) deleting the regions of the NYVAC genome encoding B8R (SEQ ID NO. 1) and B19R (SEQ ID NO. 3); and,
f) inserting into the NYVAC genome at least one polynucleotide encoding a coronavirus immunogen.

18. The method of claim 17 wherein the at least one coronavirus is selected from the group consisting of SARS-CoV-2, optionally a variant thereof selected from the group consisting of alpha, beta, gamma, delta, omicron, epsilon, zeta, eta, theta, iota, kappa, lambda, mu, cluster 5, and lineage B.1.617.

19. The method of claim 17 or 18 wherein the at least one coronavirus antigen is the psfSpike polypeptide (SEQ ID NO: 31).

20. The method of claim 19 wherein the psfSpike polypeptide is encoded by SEQ ID NO: 32.

21. The method of claim 17 encoding at least one immunogen of an influenza virus of the Influenza A, optionally any one or more of Influenza A subtypes H1 through H18 and N1 through N11; Influenza B, optionally any one or more of Influenza B subtype VIA, V1A.1, V1A.2, or V1A.3; or, the Yamagata lineage, optionally any one or more of the Y1, Y2, or Y3 subtypes.

22. The method of claim 21 wherein the polynucleotide encodes at least one influenza hemagglutinin, GenBank AY555150, neuraminidase, nucleoprotein, GenBank AAV35112, GenBank AAM75159, and/or extracellular domain of matrix 2 ion channel protein, and, GenBank AAV35111.
